(19) United States
(12) Patent Application Publication
(10) Pub. No.: US 2006/0014839 A1
(43) Pub. Date: Jan. 19, 2006

(54) Combination of angiotensin II receptor blocker and beta-blocker for secondary prevention of myocardial infarction

(76) Inventors: Robert M. Califf, Durham, NC (US); Malcolm MacNab, Far Hills, NJ (US); John JV MacMurray, Glasgow (GB); Marc A. Pfeffer, Chestnut Hills, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/514,681

(22) PCT Filed: May 16, 2003
(86) PCT No.: PCT/EP03/05198

§ 371 Date: Mar. 14, 2005

(30) Foreign Application Priority Data
May 17, 2002 (US) 60381548

(51) Int. Cl. A61P 9/10; A61K 31/00; A61K 47/00

(52) U.S. Cl. 514/789

(57) ABSTRACT

The invention relates to a method of treating cardiovascular disease in patients following myocardial infarction comprising administering an effective amount of an ARB, especially valsartan, in combination within an effective amount of a beta-blocker to such patients.

Angiotensin II receptor blockers (ARBs), such as valsartan, are known as anti-hypertensive agents which selectively block the binding of angiotensin II to the A_{T1} receptor causing vasodilatation, and diminish aldosterone secretion. Beta adrenergic blocking agents (beta-blockers) compete with epinephrine for beta-adrenergic receptors and interfere with the action of epinephrine, and are thus useful, i.a. for lowering blood pressure and heart rate and reducing cardiac arrhythmias.

Combination of potent anti-hypertensive agents in patients having reduced cardiac function subsequent to myocardial infarction has been controversial because of the risk of hypotension and bradycardia resulting in heart failure. We have now discovered in large clinical studies that in there are benefits in addition to lowering blood pressure to combination therapy comprising co-administration of ARBs, especially valsartan, together with beta-blockers in a cohort of patients after myocardial infarction.

In one aspect the present invention relates to a method of treating cardiovascular disease and thereby reducing the risk of morbidity, especially stroke, and mortality in a patient following myocardial infarction, especially myocardial infarction complicated with left ventricular dysfunction or heart failure, comprising administering to such patient an effective amount of an ARB in combination with an effective amount of a beta-blocker. More specifically the patient will have sustained an acute myocardial infarction no earlier that 12 hours, and no later than 10 days after the onset of symptoms and have had evidence of heart failure and/or of left ventricular systolic dysfunction. Even more specifically, the patients may be:
Men
Women who are not of child-bearing potential. Women are considered to be of child-bearing potential unless they are using effective contraceptive methods (hormonal contraceptive or intrauterine device or barrier with spermicide), are post-hysterectomy, or are at least one year post-tubal ligation or post-menopausal.
Aged 18 years or above
Who have sustained an acute myocardial infarction (See definition below) and are no less than 12 hours, and no more than 10 days after the onset of symptoms
Who have either clinical or radiological signs of heart failure and/or evidence of left ventricular systolic dysfunction (See definitions below.)

The method may optionally further comprise co-administration of one or more additional anti-hypertensive agents, for example an angiotensin converting enzyme inhibitor (ACEI) and/or a diuretic. In one embodiment, the patient is a normotensive patient, or a patient whose blood pressure is adequately controlled by administration of an ARB or beta-blocker alone or in combination with an additional anti-hypertensive agent other than a beta-blocker or an ARB.

ARBs suitable for use in this invention are AT₁-receptor antagonists (also called angiotensin II receptor antagonists) which bind to the AT₁-receptor subtype of angiotensin II receptor but do not result in activation of the receptor. As a consequence of the inhibition of the AT₁ receptor, these antagonists can, for example, be employed as antihypertensives or for treating congestive heart failure.

The class of AT₁ receptor antagonists comprises compounds having differing structural features, although the non-peptidic ones are preferred, e.g., compounds which are selected from the group consisting of valsartan, losartan, candesartan, eprosartan, irbesartan, saprisartan, tasosartan, telmisartan, the compound with the designation E-1477 of the following formula 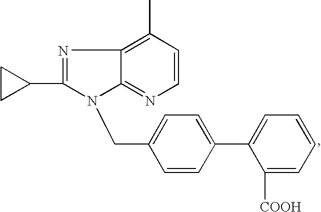
the compound with the designation SC-52458 of the following formula 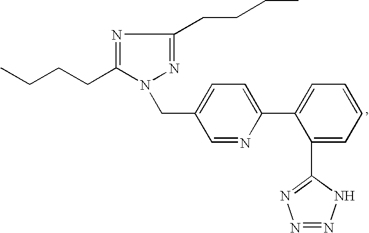
and the compound with the designation the compound ZD-8731 of the following formula

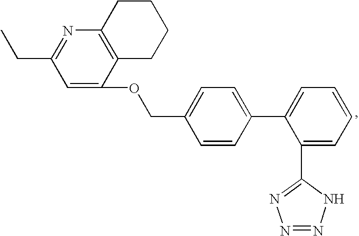

Where the ARB is an acid or base or otherwise capable of forming pharmaceutically acceptable salts or prodrugs, these forms are considered to be encompassed herein, and it is understood that such compounds may be administered in free form or in the form of a pharmaceutically acceptable salt or prodrug, such as a physiologically hydrolizable and acceptable ester.

Preferred AT₁-receptor antagonist are valsartan, losartan, candesartan, irbesartan, and telmisartan and eprosartan. Most preferred is valsartan or a pharmaceutically acceptable salt thereof. While the precise dosage will vary depending on the individual patient, and some adjustment by the treating physician may be required, suitable dosages are generally as known in the art for the compounds for use in monotherapy. For example, in the method of the invention, valsartan is preferably administered to adult patients once or twice daily for a total daily dosage of 20-320 mg, preferably 80-320 mg, preferably as the free acid. Losartan is preferably administered to adult patients orally once or twice daily, for a total daily dose of 25-100 mg, preferably as the potassium salt. Candesartan is preferably administered to adult patients at a total daily dosage of 2-32 mg, preferably in the form of its cilexetil ester. Irbesartan is preferably administered to adult patients at a total daily dosage of 150-300 mg. Telmisartan is preferably administered to adult patients at a total daily dosage of 40-80 mg, preferably as the free acid. Eprosartan is preferably administered to adults at a total daily dosage of 400-800 mg, preferably as the mesylate salt.

Beta-blockers suitable for use in the present invention include beta adrenergic blocking agents (beta-blockers) which compete with epinephrine for beta-adrenergic receptors and interfere with the action of epinephrine. Preferably, the beta-blockers are selective for the beta adrenergic receptor as compared to the alpha adrenergic receptors, and so do not have a significant alpha-blocking effect. Suitable beta-blockers include compounds selected from acebutolol, atenolol, betaxolol, bisoprolol, carteolol, esmolol, labetalol, metoprolol, nadolol, oxprenolol, penbutolol, pindolol, propranolol, sotalol, and timolol. Where the beta-blocker is an acid or base or otherwise capable of forming pharmaceutically acceptable salts or prodrugs, these forms are considered to be encompassed herein, and it is understood that the compounds may be administered in free form or in the form of a pharmaceutically acceptable salt or a prodrug such as a physiologically hydrolizable and acceptable ester. For example, metoprolol is suitably administered as its tartrate salt, propranolol is suitably administered as the hydrochloride salt, and so forth. While the precise dosage will vary depending on the individual patient, and some adjustment by the treating physician may be required, suitable dosages are generally as known in the art for the compounds for use in monotherapy. For example, suitable daily dosages for adults of the following compounds for oral administration are as indicated: acebutol—200-1200 mg; atenolol—25-100 mg; betaxolol—10-20 mg; bisoprolol—5-10 mg; carteolol—2.5-10 mg; labetalol—100-1800 mg; metoprolol—50-450 mg; nadolol—10-240 mg; oxprenol—60-480 mg; penbutolol—20-80 mg; pindolol—10-60 mg; propranolol—40-320 mg 9 or 60-320 for long-acting formulation); sotalol—160-320 mg; timolol—20-60 mg. Especially preferred beta-blockers for use in the present invention are atenolol, metoprolol and propranolol.

Other anti-hypertensive agents which may be administered in addition to the ARB and beta-blocker in the method of the invention include ACEIs and/or diuretics. Suitable ACEIs for use in the present invention include benazepril, captopril, cilazapril, enalapril, enalaprilat, fosinopril, lisinopril, moexipril, perindopril, quinapril, ramipril, and trandolapril, all in free or pharmaceutically acceptable salts. Especially preferred ACEIs for use in the present invention are benazepril, captopril, enalapril, quinapril and lisinopril, all in free or pharmaceutically acceptable salt form, for example benazepril HCl or enalapril maleate. Suitable diuretics include thiazide and related sulfonamide diuretics, for example bendroflumethiazide, benzthiazide, chlorothiazide, chlorthalidone, cyclothiazide, hydrochlorothiazide, hydroflumethiazide, indapamide, methyldiothiazide, metozalone, polythiazide, quinethazone, and trichlormethiazide; loop diuretics, for example bumetamide, ethacrynic acid, furosemide; and potassium sparing diuretics, for example amiloride, spironolactone, and triamterine. Especially preferred diuretics for use in the present invention are thiazides, especially hydrochlorothiazide.

The structure of the active agents identified by generic or tradenames may be taken from the actual edition of the standard compendium “The Merck Index” or from databases, e.g. LifeCycle Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

The invention relates to a combination, especially a pharmaceutical composition, of an angiotensin II receptor blocker (ARB) or a pharmaceutically accepatable salt thereof and a beta-blocker or a pharmaceutically acceptable salt thereof. Furthermore, the combination may comprise a further anti-hypertensive agent as described above.

The invention relates to a pharmaceutical composition, of an angiotensin II receptor blocker (ARB) or a pharmaceutically accepatable salt thereof and a beta-blocker or a pharmaceutically acceptable salt thereof for the treatment of a condition or disease as described hereinbefore or hereinafter, especially for treating cardiovascular disease in patients following myocardial infarction.

The invention relates to the use of a combination of an angiotensin II receptor blocker (ARB) or a pharmaceutically accepatable salt thereof and a beta-blocker or a pharmaceutically acceptable salt thereof (and optionally a further antihypertensive agent) in the preparation of a medicament for the treatment of a condition or disease as described hereinbefore or hereinafter, especially for treating cardiovascular disease in patients following myocardial infarction.

The pharmaceutical compositions for use in the present invention are preferably compositions for oral administration as are known and commercially available from the manufacturers. Suitable compositions and information concerning suitable pharmaceutically effective dosages and potential side effects are described in the Physician's Desk Reference. The precise dosage of the active compounds can depend on a variety of factors, such as mode of administration, age and/or individual condition. Where an active agent is an acid or base or otherwise capable of forming pharmaceutically acceptable salts or prodrugs, these forms are considered to be encompassed herein, and it is understood that the compounds may be administered in free form or in the form of a pharmaceutically acceptable salt or a prodrug such as a physiologically hydrolizable and acceptable ester, especially where the salt or prodrug form is the form approved by the regulatory authorities and commonly available.

Valsartan is supplied in the form of suitable dosage unit form, for example, a capsule or tablet, in free or pharmaceutically acceptable salt form, comprising a therapeutically effective amount, e.g. an amount equivalent to from about 20 to about 320 mg of valsartan as free acid. The administration of the active ingredient may occur up to three times a day, starting e.g. with a daily dose of 20 mg or 40 mg of valsartan, increasing via 80 mg daily and further to 160 mg daily up to 320 mg daily. Preferably, valsartan is administered once a day or twice a day to patients with a dose of 80 mg or 160 mg, for a total daily dose of 20-320 mg, preferably 80-320 mg/day. Corresponding doses may be taken, for example, in the morning, at mid-day or in the evening.

The following examples illustrate the above-described invention; however, it is not intended to restrict the scope of this invention in any manner. All publications and patents mentioned herein are incorporate by reference in their entirety as if set forth in full herein.

### EXAMPLE 1

### Early Use of Beta-Blockers in Complicated Myocardial Infarction

The early use of beta-blockers (BB) in myocardial infarction (MI) complicated by left ventricular dysfunction (LVD) or heart failure (HF) has been controversial, especially with high-dose blockade of the renin-angiotensin system (RAS).
Methods: The VALIANT trial randomized 14,808 patients (pts) with acute MI and HF/LVD to valsartan, captopril, or both. The use of BB was not mandated. We divided patients by BB use at randomization (post-MI) and at hospital discharge and compared baseline factors and outcomes.
Results: In all, 9379 were taking BB at randomization and discharge, 788 only at randomization, 1170 only at discharge, and 3045 at neither time. The BB-treated patients were somewhat younger on average, but the groups otherwise did not differ. Mortality from discharge to 30 days after enrollment was lower in patients treated with BB.

Conclusion: Although the trial did not mandate the use of BB, the rate of use was high and associated with reduced mortality, even with high-dose blockade of RAS.

B-Blocker Use

| Randomization | Yes | No | Yes | No |
|---|---|---|---|---|
| Discharge | Yes | Yes | No | No |
| Age, y* | 63 | 66 | 66 | 68 |
| Men, % | 71 | 69 | 67 | 66 |
| SBP, mm Hg* | 122 | 125 | 122 | 124 |
| Anterior MI, % | 61 | 57 | 61 | 55 |
| Diabetes, % | 21 | 27 | 24 | 27 |
| Ejection fraction, %* | 36 | 35 | 35 | 35 |
| Stroke, % | 0.6 | 0.9 | 0.9 | 1.0 |
| Death, % | 1.5 | 2.3 | 3.3 | 3.7 |

*Median.

### EXAMPLE 2

### Treatment with Valsartan, Captopril and Their Combination in High-Risk Patients After Myocardial Infarction

This example exemplifies the protocol which was used to carry out the procedure of example 1 above.

1. Introduction

The survival benefit for the use of angiotensin-converting enzyme (ACE) inhibitors in patients with acute and chronic myocardial infarction (MI) has been established by a series of internationally conducted, randomized, controlled clinical studies involving over 100,000 patients (1-4). The effectiveness of these agents in reducing mortality and the incidence of serious nonfatal cardiovascular events have been so well documented that the use of an ACE inhibitor in acute MI is now strongly endorsed by the major international cardiovascular societies (5, 6). When the totality of the evidence is considered, the overall experience indicates that this new use of an ACE inhibitor in patients with MI produces benefits that are additive to those which can be achieved with other proven therapies, such as aspirin, beta-adrenergic blockers, and reperfusion strategies (7).

Early (<24 hours) use of an orally administered ACE inhibitor in non-selective short-term studies (systolic blood pressure over 100 mm Hg) resulted in the saving of approximately 5 lives per 1000 patients treated during a 4-to 6-week course (2). In these short-term, non-selective studies the mortality benefit of ACE inhibition was greater in higher-risk patients (Killip Class 2 or greater, anterior infarcts). The trials of ACE inhibitor therapy in MI that selected for high-risk patients for more sustained therapy durations (2-4 years) produced consistent results with even more impressive benefits of ACE inhibitor therapy (4). For mortality alone, the lives saved in these selective studies ranged from 40 to 76 per 1000 patients treated. Each of these long-term studies also demonstrated other important clinical benefits of this use of ACE inhibitors in reducing major nonfatal cardiovascular events. The selection criteria used to identify higher-risk patients in the SAVE study (8) was a left ventricular ejection fraction of 40% or less whether or not transient signs of pulmonary congestion were present or absent (40 and 60%, respectively). In the AIRE study (9), patient selection was for clinical, even transient, evidence of heart failure. In the TRACE study (10), echocardiographic wall motion abnormalities were used to identify a higher risk population. Despite these proven benefits of ACE inhibitor therapy in patients with MI, a substantial proportion of patients experience major cardiovascular complications, including death while on this therapy. The newly developed angiotensin II receptor blockers (ARB), as specific inhibitors of the final step in the renin-angiotensin cascade, can provide an opportunity to more completely inhibit this system pharmacologically. Two studies raised the question of whether this new pharmacologic modality for inhibiting the renin-angiotensin system may offer specific advantages to patients with myocardial infarction: the first was demonstration of local generation of angiotensin II independent of angiotensin converting enzyme; the second was the demonstration that plasma levels of angiotensin II often return to pretreatment values during long-term ACE inhibition therapy. On the other hand, augmentation of bradykinin secondary to the ACE inhibitors reduction in the degradation of this vascularly-active compound may offer additional clinical advantages that cannot be anticipated from the use of the ARB (II-12). However, this same accumulation of bradykinin has been associated with side effects such as cough, which have led to discontinuation of ACE inhibition therapy (13). Whether ARBs provide at least comparable clinical effectiveness with better tolerability or blockade at the receptor level will only be determined by appropriate clinical studies.

The Evaluation of Losartan In The Elderly (ELITE) trial has generated preliminary evidence to support the position that fuller inhibition of the renin-angiotensin system by an ARB may lead to greater clinical benefits (14). The ELITE investigators identified elderly patients with heart failure who had not been previously treated with an ACE inhibitor. These patients were randomized to standard doses of captopril (50 mg, t.i.d.) or treatment with the ARB, losartan, in a double-blind manner. The primary objective of this 722 patient study was to compare the tolerability and increase in serum creatinine with this ARB versus the ACE inhibitor. Although there were no significant differences between therapies in the primary objective, a statistically significant reduction in all-cause mortality was found with the use of losartan. This difference in survival was based on a total of only 49 deaths and therefore must be considered as preliminary and hypothesis generating. Nonetheless, this initial direct comparison between an ACE inhibitor and an ARB provides support for the inhibition of the angiotensin system at the receptor rather than at the converting enzyme level.

Another approach worthy of investigation is the use of a combination of an ACE inhibitor and an ARB to offer the potential advantages of more complete inhibition of the renin-angiotensin-system by action at two points in the pathway to reduce the effects of angiotensin II while sustaining the potential benefits of the augmentation of bradykinin produced by ACE inhibitors. The potential, therefore, to demonstrate a further improvement in the care of patients with myocardial infarction with the use of an ARB alone or in combination with an ACE inhibitor provides the major rationale for the current study. The purpose of this investigation is to determine whether the ARB, valsartan, is more effective or at least as effective and better tolerated as a proven ACE inhibitor in the reduction of mortality in higher-risk MI patients (AIRE, SAVE and TRACE criteria) and to ascertain whether the addition of valsartan to a proven ACE inhibitor regimen will result in an even greater reduction in mortality than achieved with the ACE inhibitor monotherapy alone.

2. Study Objectives

Primary Objectives:

To demonstrate that long-term administration of valsartan given as monotherapy is more effective than captopril given as monotherapy in the reduction of total mortality after an acute myocardial infarction.

To demonstrate that long-term administration of the combination of valsartan with captopril is more effective than captopril given as monotherapy in the reduction of total mortality after an acute myocardial infarction.

If valsartan as monotherapy cannot be shown to be superior to captopril as in objective 1, to demonstrate that long-term administration of valsartan given as monotherapy is at least as effective as captopril given as monotherapy in the reduction of total mortality after an acute myocardial infarction.

Secondary Objective:

To demonstrate that long-term administration of the combination of valsartan with captopril is more effective than valsartan given as monotherapy in the reduction of total mortality after an acute myocardial infarction.

Other Key Parameters:

To compare the resource utilization and quality of life of the three treatment groups.

To compare the safety and tolerability of the three treatment arms.

3. Investigational Plan

3.1. Overall Study Design

Study Design

VALIANT is a prospective multinational, multicenter, double-blind, randomized, active-controlled phase III study with three parallel treatment groups.

Patient Population

The study population will consist of patients who have sustained an acute myocardial infarction and are randomized no earlier than 12 hours, and no later than 10 days after the onset of symptoms. Patients will also have evidence of heart failure and/or left ventricular systolic dysfunction. (Also see Section 3.3: Study population.)

Sample Size

A total of 14,500 patients, allocated in a 1:1:1 ratio to captopril monotherapy, valsartan monotherapy, or the combination of valsartan and captopril, respectively, will be randomized. (Also see Section 6.2: Sample size and power considerations.)

Study Treatments

The three treatment groups are (See FIG. 3.1-1: Treatment regimen and Section 3.4.1: Investigational therapy and reference therapy.):
1. Captopril monotherapy (active control drug). The target dose is 50 mg three times daily.
2. Valsartan monotherapy (investigational drug). The target dose is 160 mg twice daily.
3. The combination of captopril and valsartan (investigational regimen). The target doses are 50 mg three times daily and 80 mg twice daily, respectively.

The objective of treatment is to ensure that each patient receives the maximal tolerated dose of study medication up to the target dose. Study medication is administered in a stepwise titration with four titration steps (Steps I-IV).

The titration should follow the recommendations and criteria described in Section 3.4.1: Investigational therapy and reference therapy, but the decision whether or not to up-titrate is left to the investigator's discretion depending upon the patient's status.

Patients are to be treated from the day of randomization to the end of the study except in case of temporary interruption or permanent discontinuation, as described in Section 3.3.3: Interruption or discontinuation of treatment.

[image]

Study Duration

The study duration is variable and depends upon achieving a pre-specified number of primary efficacy endpoints, deaths. Unless completed early because of a statistically significant interim analysis or a safety concern, the study will continue until 2700 patients have reached the primary endpoint, death. On the date that number of deaths is achieved, the vital status of all randomized patients will be collected and the study will be considered completed as described in Section 6.2: Sample size and power considerations.

For planning purposes, the expected study duration is approximately 4 years including an enrollment period of 18 months. In reality, the actual study duration will depend upon the actual accrual rate, the length of the accrual period, and the observed death rate. The study duration may, therefore, be shorter or longer than 4 years. In case the required number of events has not been observed after a study duration of 6 years, however, the study will be closed and considered completed.

3.2. Discussion of Design

This study is designed to test whether inhibition of the renin-angiotensin system with valsartan, an ARB, will be more effective than, or at least as effective as, captopril, an ACE inhibitor, and whether the combination of an ACE inhibitor and valsartan is more effective than an ACE inhibitor alone in the reduction of total mortality in high risk patients with an acute myocardial infarction.

As outlined in the introduction, although there is some debate as to whether all patents with acute myocardial infarction should receive early treatment with ACE inhibition, there is overwhelming evidence that ACE inhibitors reduce mortality and morbidity after myocardial infarction in patients with evidence of heart failure and/or left ventricular systolic dysfunction (5-6). Such high-risk patients should receive this therapy commencing early and maintained long term (1-7).

As a result, it is unlikely for ethical reasons that additional placebo-controlled trials of ACE inhibitors will be conducted in such patients (15). VALIANT, therefore, requires an active-control reference treatment and, consequently, external validation as defined in the ICH guidelines (16).

The AIRE, SAVE and TRACE studies (8-10) have been chosen for external validation since they are the definitive placebo-controlled mortality long-term studies that have defined, on the basis of survival benefit, the high-risk patient population with myocardial infarction who should receive long-term ACE inhibitor therapy. The results of these studies were homogeneous and consistent not only for the primary endpoint of all-cause mortality (OR 0.74, 95% Cl 0.66 to 0.83 for the 3 studies pooled and OR of 0.79, 0.70 and 0.73 for AIRE, SAVE and TRACE, respectively) but also clinically important non-fatal endpoints such as time to first hospitalization for congestive heart failure (OR 0.73, 95% Cl 0.63 to 0.85 for the 3 studies pooled and OR of 0.74, 0.65 and 0.78 respectively) and time to first recurrent myocardial infarctions (OR 0.80, 95% Cl 0.69 to 0.94 for the 3 studies pooled and OR of 0.89, 0.80 and 0.75 respectively). A common feature of AIRE, SAVE and TRACE is the identification of high-risk patients either by signs and symptoms of heart failure and/or by objective measurement of left ventricular systolic dysfunction. High-risk patients will be selected in VALIANT (also a long-term study) using the same inclusion criteria.

There are interesting similarities regarding the effect of ACE inhibitors on all-cause mortality in placebo-controlled studies in high-risk patients with myocardial infarction and in patients with congestive heart failure. In AIRE, SAVE and TRACE (4) there were 1568 deaths from 5966 randomized patients and an odds ratio for all-cause mortality of 0.74 (Cl: 0.66 to 0.83). In a meta-analysis of placebo-controlled studies in patients with congestive heart failure, there were 1320 deaths from 7105 randomized patients (17) and an odds ratio for all-cause mortality of 0.77 (Cl: 0.67 to 0.88). The benefits of ACE inhibitors on all-cause mortality in patients with impaired cardiac function, whether it is stable symptomatic heart failure or left ventricular dysfunction following a myocardial infarction, are therefore quite comparable and well quantified.

The ACE inhibitor chosen for comparison has a well-documented efficacy and safety profile and an established dosage regimen. In the overall experience of ACE inhibitors in acute and chronic infarction, captopril was used in the non-selective early studies ISIS-4 and Chinese Captopril Study (18, 19) as well as in the selective long-term study SAVE (8), resulting in the largest cumulative experience with an ACE inhibitor in controlled clinical trials. Since captopril was effective with both early and long-term administration, a safe and effective dosage regimen is available for comparison with valsartan.

VALIANT, therefore, is a pragmatic study (20) that reflects optimal current clinical practice and treatments. The treating physicians are encouraged to employ optimal (i.e., life-saving) standard treatments (e.g., aspirin, thrombolytics or primary angioplasty and beta-blockers) in their patients. They are further encouraged to randomize patients into VALIANT who would usually be considered for treatment with an ACE inhibitor, i.e., with evidence of heart failure and/or left ventricular systolic dysfunction. The identification of such high-risk patients is based closely on the criteria used in the three relevant large clinical studies, AIRE, SAVE and TRACE. As is the case with these three studies, VALIANT is a long-term study with all cause mortality as primary efficacy endpoint. The active-control ACE inhibitor is captopril using the dosage regimen evaluated in SAVE.

3.3. Study Population

3.3.1 Patient Population

The patient population will consist of patients who have sustained an acute myocardial infarction and are randomized no earlier than 12 hours, and no later than 10 days after the onset of symptoms. Patients must also have evidence of heart failure and/or of left ventricular systolic dysfunction.

A total of 14,500 patients is to be included in this study, with approximately 4833 patients in each of the three treatment arms. (Also see Section 6.2: Sample size and power considerations.)

3.3.2. Inclusion and Exclusion Criteria

Inclusion Criteria

The following patients may qualify for inclusion in the study:
Men
Women who are not of child-bearing potential. Women are considered to be of child-bearing potential unless they are using effective contraceptive methods (hormonal contraceptive or intrauterine device or barrier with spermicide), are post-hysterectomy, or are at least one year post-tubal ligation or post-menopausal.
Aged 18 Years or Above

Who have sustained an acute myocardial infarction (See definition below.) and are no less than 12 hours, and no more than 10 days after the onset of symptoms
Who have either clinical or radiological signs of heart failure and/or evidence of left ventricular systolic dysfunction (See definitions below.)
   Definitions
   Acute Myocardial Infarction:

In order to fulfill the criteria for an acute myocardial infarction:
All patients must have an increase in the plasma concentration of cardiac enzymes. Either of the following will fulfill the requirement for an increase in cardiac enzymes:
Total creatine-kinase (CK) at least 2 times the upper limit of the normal range, or CK-MB above the upper limit of the normal range and at least 5% of the total CK
Note: If total CK or CK-MB is not available, the following will be accepted in fulfillment of the criteria for acute myocardial infarction:
Troponin T at least 3 times the upper limit of the normal range
Troponin I at least 3 times the upper limit of the normal range
Other cardiac enzymes are not considered adequate.
All patients must also have either a typical clinical presentation and/or typical ECG changes.
Typical ECG changes include evolving ST-segment or T-wave changes in two or more contiguous ECG leads, the development of new pathological Q/QS waves in two or more contiguous ECG leads, or the development of new left bundle branch block.
   Heart Failure:

Heart failure is defined by at least one of the following criteria:
Radiological evidence of left ventricular failure. This is defined as pulmonary venous congestion with interstitial or alveolar edema and must be supported by at least one chest radiograph.
Clinical evidence of left ventricular failure. This is defined as pulmonary edema (bilateral post-tussive crackles extending at least one-third of the way up the lung fields in the absence of pulmonary disease) or the presence of a third heart sound with a persistent tachycardia.
Clinical or radiological evidence of heart failure following the qualifying acute myocardial infarction can be transient and need not necessarily be present at the time of randomization.
   Left Ventricular Systolic Dysfunction

At least one of the following will be considered sufficient evidence of left ventricular systolic dysfunction:
1. Echocardiography: left ventricular ejection fraction (LVEF)≦35% or a wall motion index≦1.2
2. Radionuclide ventriculography: LVEF≦40%
3. Ventricular contrast angiography: LVEF≦35%.

None of these examinations is mandatory for this study but may be performed as part of standard care. No central measurement by a core laboratory is required for this study.

Exclusion Criteria

At the time of randomization, none of the following may exist:
Failure to provide informed consent
Cardiogenic shock (within the 24 hours prior to randomization)
Systolic blood pressure <100 mm Hg
Serum creatinine >221 μmol/L (2.5 mg/dl) (most recent value obtained after the qualifying myocardial infarction and before randomization)
Known or suspected bilateral renal artery stenosis
Stroke or transient ischemic attack within the previous one month
Refractory potentially lethal ventricular arrhythmia
Refractory angina
Cardiac surgery planned to occur within the 15 days after randomization
Known intolerance of, or contra-indication to, an ACE inhibitor or angiotensin receptor blocker
Clinically significant right ventricular qualifying myocardial infarction
Pre-existing valvular heart disease likely to require surgery within the next three months
Obstructive cardiomyopathy
Serious non-cardiovascular disease severely limiting life expectancy
Pregnant or nursing women
Previous major organ (e.g., lung, liver, heart, kidney) transplantation or on transplant waiting list

Other conditions/circumstances likely to lead to poor treatment adherence (e.g., history of poor compliance, alcohol or drug dependency, psychiatric illness, no fixed abode)
Current participation in another clinical trial in which a patient is currently taking an investigational drug. A patient in the follow-up period of another clinical trial but no longer taking the investigational drug, or patients in a clinical trial with a drug already registered in this indication could be considered for inclusion in the study if in accordance with local regulations and advance permission from Novartis has been obtained.
Current participation in another clinical trial with an investigational medical device except for non-coated or heparin-coated stents.
Note: Treatment with an ACE inhibitor or an angiotensin II blocker prior to randomization is not an exclusion, provided this treatment is discontinued at least 12 hours before randomization.
   Change in Cardiac Marker Criteria in Amendment 2

In order to fulfill the criteria for an acute myocardial infarction:
All patients must have an increase in the plasma concentration of cardiac enzymes. Any of the following will fulfill the requirement for an increase in cardiac enzymes:
If both total creatine-kinase (CK) and CK-MB are available, total CK must be at least 2 times the upper limit of the normal range, and CK-MB must be above the upper limit of the normal range and at least 5% of the total CK
If only total creatine-kinase (CK) is available, total CK must be at least 2 times the upper limit of the normal range
If only CK-MB is available, CK-MB must be at least 2 times the upper limit of the normal range.
If neither total CK nor CK-MB are available, the following markers will be accepted in fulfillment of the criteria for acute myocardial infarction:
Troponin T at least 3 times the upper limit of the normal range
Troponin I at least 3 times the upper limit of the normal range
Other cardiac enzymes are not considered adequate.
All patients must also have either a typical clinical presentation and/or typical ECG changes.
Typical ECG changes include evolving ST-segment or T-wave changes in two or more contiguous ECG leads, the development of new pathological Q/QS waves in two or more contiguous ECG leads, or the development of new left bundle branch block.
   Change in Cardiac Marker Criteria in Amendment 3

In order to fulfill the criteria for an acute myocardial infarction:
All patients must have an increase in the plasma concentration of appropriate markers of cardiac necrosis. Any of the following will fulfill the requirement for an increase in cardiac markers:
If both total CK and CK-MB are above the upper limit of normal (>ULN) and either total CK or CK-MB are at least twice the upper limit of normal
If CK-MB is elevated to at least twice the upper limit of normal (2×ULN) when total CK is not available, or to above the ULN if confirmed by an accompanying Troponin T or I level at least three times the upper limit of normal (3×ULN)
If total CK is elevated to at least twice the upper limit of normal (2×ULN) when CK-MB is not available, or to above the ULN if confirmed by an accompanying Troponin T or I level at least three times the upper limit of normal (3×ULN)
If Troponin T or I level is at least five times the upper limit of normal (5×ULN) and neither total CK nor CK-MB are available

Thus, patients having any of the eight sets of values summarized in the table below will fulfill the cardiac marker criteria for this trial:

| CK | CK-MB | TROP |
|---|---|---|
| >ULN | ≧2xULN | — |
| ≧2xULN | >ULN | — |
| >ULN | >ULN | ≧3xULN |
| NA | ≧2xULN | — |
| — | >ULN | ≧3xULN |
| ≧2xULN | NA | — |
| >ULN | — | ≧3xULN |
| NA | NA | ≧5xULN |

NA = Not Available

Other cardiac markers are not considered adequate.
All patients must also have either a typical clinical presentation and/or typical ECG changes.
Typical ECG changes include evolving ST-segment or T-wave changes in two or more contiguous ECG leads, the development of new pathological Q/QS waves in two or more contiguous ECG leads, or the development of new left bundle branch block.

Collated Cardiac Markers Chart Including All Potential Cardiac Marker Criteria

| CK | CK-MB | TROP |
|---|---|---|
| >ULN | ≧2xULN | — |
| ≧2xULN | >ULN | — |
| >ULN | >ULN | ≧3xULN |
| NA | ≧2xULN | — |
| — | >ULN | ≧3xULN |
| ≧2xULN | NA | — |
| >ULN | — | ≧3xULN |
| NA | NA | ≧5xULN |
| — | >ULN and ≧5% | — |
| | of total CK | |
| >ULN | >ULN and ≧5% | — |
| | of total CK | |
| NA | NA | ≧3XULN |

NA = Not Available

3.3.3. Interruption or Discontinuation of Treatment

Every effort must be made to ensure that patients remain in the study and on study medication for the duration of the study. Each randomized patient must be followed until study completion whether or not the first dose of study medication is taken, or study medication is temporarily interrupted or permanently discontinued. A patient is considered randomized when the patient identification number has been assigned by the automated randomization system, Q-tone (See Section 3.4.2: Treatment assignment.).

If either the study medication or observations of a patient are discontinued, the reason(s) for the discontinuation are to be collected and recorded in the CRF.

Temporary Interruption of Study Medication

A temporary interruption of study medication may occasionally be required. If a temporary interruption occurs, the Coordinating Center Medical Hot Line should be notified and study medication should reinitiated as soon as possible. Every attempt to reinitiate study medication should be made throughout the duration of the study. The reinitiation of study medication is not subject to a time limit, and the number of attempts to reinitiate medication is not limited.

When study medication is reinitiated, it is not necessary to begin at the lowest dose. Study medication may be restarted at the previously administered dose, or at any of the titration steps, at the investigator's discretion depending on the patient's clinical status.

Patients with temporary interruptions of study medication should continue to follow the visit schedule and be evaluated for the occurrence of endpoints.

Study medication must be interrupted for pregnancy, for the duration of gestation and lactation.

Permanent Discontinuation of Study Medication

A permanent discontinuation of study medication may be considered only when one of the following conditions exist:
A patient decides it is in his or her best interest, i.e., withdraws his or her consent
An investigator considers it advisable for a sound clinical reason and after discussion with the Coordinating Center Medical Hot Line
An intolerable adverse experience occurs that is suspected to be related to study medication or that prevents the patient's continuation of study medication
A life-threatening adverse experience or laboratory abnormality occurs that is suspected to be related to study medication
A patient's study medication is unblinded.

Whenever possible, patients will not be permanently discontinued from study medication without prior discussion with the Coordinating Center. Treatment options will be discussed, and if a permanent discontinuation is decided, alternate therapy should be instituted. Patients who are permanently discontinued from study medication should continue the visit schedule and undergo evaluation for the occurrence of endpoints. All procedures should be completed as specified except for the documentation of study medication returns and dispensing. These patients may not enroll in any subsequent investigational drug or device studies without permission from the Executive Committee until this study ends.

In cases where the patient has withdrawn consent, at least vital status, as a matter of public record in most countries, will be followed to the end of the study.

Discontinuation from the Study

A patient will be considered discontinued from the study only if he or she is lost to follow-up after exhausting all means of contact.

If a patient is definitively lost to follow-up, the status of the patient at the last visit or contact will be used for the final analysis.

3.4. Treatments

3.4.1. Investigational Therapy and Reference Therapy

Description

Novartis will supply all study medication.

Valsartan (investigational therapy) will be supplied in the form of 20 mg, 40 mg, 80 mg, and 160 mg capsules. Matching placebo capsules will be provided to maintain the blinded dose regimen.

For all but the first distribution of study medication, captopril (reference therapy) will be supplied in the form of 6.25 mg, 12.5 mg, 25 mg, and 50 mg tablets. The captopril 6.25 mg tablet will be manufactured by Novartis based on the commercial 12.5 mg tablet formulation from Azupharma Gmb H & Co. (Germany). The captopril 12.5, 25, and 50 mg tablets will be obtained as commercial supplies from Azupharma. Matching placebo tablets, manufactured by Novartis, will be provided to maintain the blinded dose regimen.

At the start of the study, captopril will be supplied in the form of 6.25 mg, 12.5 mg, 25 mg, and 50 mg capsules with matching placebo capsules to maintain the blind. These capsules, manufactured by Novartis contain Azupharma captopril tablets that have been crushed and encapsulated to match the valsartan capsules. These capsules, however, have a shelf life of only one year after manufacture. This one-year shelf life is not practical to conduct a study of the size and duration of VALIANT. Therefore, the captopril supplies provided for all but the first supply distribution (approximately 1000 patients) will consist of the Azupharma commercial tablets. In vitro dissolution testing has been conducted and the results indicated equivalence of the captopril capsules and tablets.

Note: In the remainder of the study drug supply discussion, the supplies description will include valsartan capsules and captopril tablets. Asterisks will be used to denote when the initial supplies will contain captopril capsules instead of tablets.

Packaging

Study medication will be packaged in blisters. Each blister will contain 21 capsules of valsartan and 21 tablets (*capsules) of captopril, which is sufficient for seven days of treatment. There will be seven numbered columns and three rows of pockets on each blister.

The columns will be numbered from 1 to 7 corresponding to the seven days of the week. The rows will be labeled to correspond to the morning, mid-day, and evening doses.

The blisters will be labeled with color-coded labels, one color for each of the four titration steps.

Two types of study medication packs will be provided: titration packs and 4-month treatment packs, as described in the following table.

TABLE 3.4.1-1
Study medication packs

| Pack type | Use | Description of contents |
|---|---|---|
| Titration | 1. Initial dose titration | One carton containing 8 blisters, |
| Pack | 2. If retitration is needed | two color-coded blisters for each |
| | during the study, for | for the four titration steps (I, II, III, |
| | example, after down- | and IV). |
| | titration or temporary | Each blister contains sufficient |
| | interruption of study | study medication for seven days |
| | medication. | |
| 4-month | Maintenance dose | One color-coded carton containing |
| treatment | | sufficient study medication for 4 |
| pack | | months (20 weeks) of treatment at |
| | | titration Step I, Step II, Step III, or |
| | | Step IV. |
| | | Each 4-month treatment pack |
| | | contains four monthly treatment |
| | | packs. Each monthly treatment |
| | | pack contains 5 blisters. |
| | | Each blister contains sufficient |
| | | study medication for seven days. |

Labeling

Study medication labels will comply with the legal requirements of each country, will be printed in the local language, and will contain the storage conditions.

The titration and 4-month treatment packs will contain two-part labels. One part will remain affixed to the pack and the second part will be a tear-off portion which will be attached to the CRF for documentation. Both parts of the label will contain a space for the study center to write in the patient identification information. The monthly treatment packs, contained within the 4-month treatment pack, will bear only a permanently affixed label with no tear-off portion.

Administration of Study Treatment

Each dose of study medication will consist of one valsartan or placebo capsule and one captopril or placebo tablet (*capsule). Study medication is to be swallowed with water. Doses will be taken in the morning, at mid-day, and in the evening. The patient should be instructed to take the doses at approximately the same times each day, preferably one hour before meals. The dosage scheme is presented in the following four tables.

TABLE 3.4.1-2
Step 1 dose administration

| | Morning (AM) | | | |
|---|---|---|---|---|
| | dose | Midday dose | | |
| Treatment | (# | (# | Evening (PM) dose | Total daily |
| Group | capsules/tablets) | capsules/tablets) | (# capsules/tablets) | dose |
| Valsartan | (1) 20 mg | (1) placebo | (1) 20 mg valsartan | 40 mg |
| monotherapy | valsartan capsule | capsule | capsule | |
| | (1) placebo | (1) placebo tablet** | (1) placebo tablet** | |
| | tablet** | | | |
| Captopril | (1) 6.25 mg | (1) 6.25 mg | (1) 6.25 mg | 18.75 mg |
| monotherapy | captopril tablet** | captopril tablet** | captopril tablet** | |
| | (1) placebo | (1) placebo | (1) placebo capsule | |
| | capsule | capsule | | |
| Combination | (1) 20 mg | (1) placebo | (1) 20 mg valsartan | 40 mg |
| therapy | valsartan capsule + (1) | capsule + (1) | capsule + (1) | valsartan + 18.75 mg |
| | 6.25 mg | 6.25 mg | 6.25 mg | captopril |
| | captopril tablet** | captopril tablet** | captopril tablet** | |

**Captopril and matching placebo will be supplied in capsules for the first supply distribution only. Thereafter, captopril and matching placebo will be supplied in tablets.

TABLE 3.4.1-3
Step 2 dose administration

| | Morning (AM) | | | |
|---|---|---|---|---|
| | dose | Midday dose | | Total |
| Treatment | (# | (# | Evening (PM) dose | daily |
| Group | capsules/tablets) | capsules/tablets) | (# capsules/tablets) | dose |
| Valsartan | (1) 40 mg | (1) placebo | (1) 40 mg valsartan | 80 mg |
| monotherapy | valsartan capsule | capsule | capsule | |
| | (1) placebo | (1) placebo tablet** | (1) placebo tablet** | |
| | tablet** | | | |
| Captopril | (1) 12.5 mg | (1) 12.5 mg | (1) 12.5 mg | 37.5 mg |
| monotherapy | captopril tablet** | captopril tablet** | captopril tablet** | |
| | (1) placebo | (1) placebo | (1) placebo capsule | |
| | capsule | capsule | | |
| Combination | (1) 20 mg | (1) placebo | (1) 20 mg valsartan | 40 mg |
| therapy | valsartan capsule + (1) | capsule + (1) | capsule + (1) | valsartan + 37.5 mg |
| | 12.5 mg | 12.5 mg | 12.5 mg | captopril |
| | captopril tablet** | captopril tablet** | captopril tablet** | |

**Captopril and matching placebo will be supplied in capsules for the first supply distribution only. Thereafter, captopril and matching placebo will be supplied in tablets.

TABLE 3.4.1-4
Step 3 dose administration

| | Morning (AM) | | Evening (PM) | |
|---|---|---|---|---|
| | dose | Midday dose | dose | |
| Treatment | (# | (# | (# | Total daily |
| Group | capsules/tablets) | capsules/tablets) | capsules/tablets) | dose |
| Valsartan | (1) 80 mg | (1) placebo | (1) 80 mg | 160 mg |
| monotherapy | valsartan capsule | capsule | valsartan capsule | |
| | (1) placebo | (1) placebo tablet** | (1) placebo | |
| | tablet** | | tablet** | |
| Captopril | (1) 25 mg | (1) 25 mg captopril | (1) 25 mg | 75 mg |
| monotherapy | captopril tablet** | tablet** | captopril tablet** | |
| | (1) placebo | (1) placebo | (1) placebo | |
| | capsule | capsule | capsule | |
| Combination | (1) 40 mg | (1) placebo | (1) 40 mg | 80 mg |
| therapy | valsartan capsule + (1) | capsule + (1) | valsartan capsule + (1) | valsartan + |
| | 25 mg | 25 mg captopril | 25 mg | 75 mg |
| | captopril tablet** | tablet** | captopril tablet** | captopril |

**Captopril and matching placebo will be supplied in capsules for the first supply distribution only. Thereafter, captopril and matching placebo will be supplied in tablets.

TABLE 3.4.1-5
Step 4 dose administration

| | Morning (AM) | | Evening (PM) | |
|---|---|---|---|---|
| | dose | Midday dose | dose | |
| Treatment | (# | (# | (# | Total daily |
| Group | capsules/tablets) | capsules/tablets) | capsules/tablets) | dose |
| Valsartan | (1) 160 mg | (1) placebo | (1) 160 mg | 320 mg |
| monotherapy | valsartan capsule | capsule | valsartan capsule | |
| | (1) placebo | (1) placebo tablet** | (1) placebo | |
| | tablet** | | tablet** | |
| Captopril | (1) 50 mg | (1) 50 mg captopril | (1) 50 mg | 150 mg |
| monotherapy | captopril tablet** | tablet** | captopril tablet** | |
| | (1) placebo | (1) placebo | (1) placebo | |
| | capsule | capsule | capsule | |
| Combination | (1) 80 mg | (1) placebo | (1) 80 mg | 160 mg |
| therapy | valsartan capsule + (1) | capsule + (1) | valsartan capsule + (1) | valsartan + 150 mg |
| | 50 mg | 50 mg captopril | 50 mg | captopril |
| | captopril tablet** | tablet** | captopril tablet** | |

**Captopril and matching placebo will be supplied in capsules for the first supply distribution only. Thereafter, captopril and matching placebo will be supplied in tablets.

Titration Criteria

Study medication is to be initiated at titration Step I as soon as possible after randomization.

Treatment can be started at any time during the day (morning, midday, or evening dose). Up-titration can also be carried out at any time during the day (morning, midday, or evening dose), providing the titration criteria are met.

Note: For patients who are taking captopril 25 mg t.i.d. (or the equivalent dose of another ACE inhibitor or angiotensin receptor blocker) at the time of evaluation for entry into the study and who are clinically stable, study medication may be initiated at Step 11 instead of

Step I. Previous ACE inhibitor or angiotensin receptor blocker therapy must have been withdrawn for at least 12 hours prior to randomization. If using this accelerated titration schedule, the first dose of Step II and of Step III must not be the midday dose.

The criteria for upward titration of study medication are:
Persistent systolic blood pressure ≧100 mm Hg if within 72 hours after the onset of acute myocardial infarction, or >90 mm Hg if beyond 72 after the onset of acute myocardial infarction (repeat measurements must be taken in the same position, supine, sitting, or standing)
No symptoms of hypotension, e.g., syncope, orthostatic dizziness, faintness, lightheadedness

Serum creatinine must be ≦265 μmol/L (3.0 mg/dl) and must not have increased by more than 88.4 μmol/L (1.0 mg/dl) from baseline (Visit 1 value). Step III should not be exceeded if the serum creatinine rises above 221 μmol/L (2.5 mg/dl).

Measurement and recording in the CRF of serum creatinine is required only before the initial up-titration of the study medication to Steps I, III, and IV. Otherwise, this measurement is left to the investigator's discretion according to local practice guidelines.

Recommendations for Achievement of Dose Titration Steps

As long as the patient fulfills the criteria for upward titration of study medication before any increase in the dose of study medication, the duration of treatment at each of the titration steps is at the investigator's discretion based upon the patient's status. However, up-titration to Step II should be attempted no earlier than the day after randomization (Day 2). In addition, only one up-titration should be attempted during the same day.

Note: For patients who were taking captopril 25 mg t.i.d. (or the equivalent dose of another ACE inhibitor or angiotensin receptor blocker) at the time of evaluation for entry into the study, who were clinically stable, and for whom study medication was initiated at Step II instead of Step I, the investigator has the option of advancing to Step III after a 12-hour observation period instead of waiting until the day after randomization as long as the criteria for upward titration are fulfilled.

If at all possible, the investigator should aim to titrate the dose of study medication to at least titration Step III before hospital discharge (Visit 2 for most patients). If this is not possible, the investigator should make every effort to achieve at least titration Step II. It is only acceptable to discharge a patient on titration Step I if Step II has not been tolerated or could not be given because the titration criteria were not met.

If a patient cannot tolerate titration Step I, the investigator should continue to retest this titration step throughout the study. Every effort should be made to ensure that a patient receives treatment during the study.

Up-titration should be considered at every evaluation unless the patient is currently at Step 1V or has been permanently discontinued from study medication. Not all patients will achieve Step 1V, but the objective is for all patients to have at least attempted Step 1V by the time of the three-month evaluation (Visit 4).

At any time during the study, down-titration or temporary interruption is permitted if a patient cannot tolerate a particular dose, for example, in case of symptomatic hypotension or renal impairment, or if the study medication cannot be continued for a concomitant medical condition or surgery. (Also see Section 3.3.3: Interruption or discontinuation of treatment.

Continuation of Study Medication

Study medication will not be provided after completion or early termination of the study.

3.4.2. Treatment Assignment

Patients providing informed consent and fulfilling all other inclusion and exclusion criteria will be randomly allocated to one of the three treatment groups in a 1:1:1 ratio.

Allocation of patients to treatment groups will be accomplished centrally using a 24-hour interactive voice-activated response telephone call-in system (Q-tone). Each person authorized to obtain randomization information will be assigned a site identification number (user identification) and a unique pin number. Upon the site calling Q-tone, entering the site and pin numbers, requesting to randomize a patient, and verifying the patient's eligibility, the Q-tone system will assign to the patient a three-digit patient number and identify the first drug kit to be dispensed. The combination of a four-digit site identification number and the three-digit patient number will uniquely identify the patient for the duration of the study.

A patient will be considered randomized when the Q-tone system assigns the patient three-digit identification number.

A stock of study medication treatment packs identified by Drug Code numbers will be maintained at the site. The site will call Q-tone to obtain the Drug Code number for the appropriate treatment pack to dispense to the patient.

3.4.3. Blinding

The blind will be maintained in a double dummy fashion by supplying valsartan and placebo in matching capsules, and captopril and placebo in matching tablets (*capsules). At each dose, patients will take one capsule of valsartan or placebo and one tablet (*capsule) of captopril or placebo.

Randomization will be performed by Novartis Drug Supply Management using a validated system that automates the random assignment of treatment groups to randomization numbers. The randomization scheme will be reviewed by the Quality Management Biostatistics Group in Novartis Medical Information Processing and Statistics Department and locked by them after approval.

Within the Q-tone system, the randomization numbers will be used to link the patient identification number to the correct Drug Code numbers on the treatment packs.

Randomization data are kept strictly confidential, accessible only to authorized persons, until the time of unblinding. At the conclusion of the trial, the occurrence of any emergency code breaks will be determined after return of all code break reports and unused drug supplies to Novartis. Only when the study has been completed, the data file verified, and the protocol violations determined will the drug codes be broken and made available for data analysis.

At the times of interim analyses, the treatment assignments for the patients included in that analysis will be transmitted to the independent statistician at the independent statistical center. Data will be presented to the DSMB in a semiblinded manner (Treatments A, B, and C). The DSMB statistician will possess a sealed copy of the treatment decode scheme for unblinding purposes if unblinding is deemed necessary by the DSMB. In such cases, unblinding of the DSMB will be documented in the minutes. The DSMB minutes will be made available only after the trial has been completed and the data analyzed.

For details of the emergency procedure for unblinding of individual patients in cases of emergency, see Section 9.1.2.

3.4.4. Concomitant Therapy

Every effort must be made to avoid the use of the following medications at any time during the study:

ACE Inhibitors Other than Study Medication

Angiotensin receptor blockers other than study medication.

In exceptional circumstances, the investigator may feel it is necessary to substitute open-label ACE inhibitor or angiotensin receptor blocker therapy for study medication. Such a course of action should only very rarely be necessary and is, in general, strongly discouraged. Before any treatment with open-label ACE inhibitor or angiotensin receptor blocker is initiated, the situation must be discussed with the study's Coordinating Center Medical Hot Line staff. Any agreed upon period of open-label therapy must be kept to the minimum length of time necessary, and study drug treatment should be reinstituted as soon as possible thereafter.

Patients taking an ACE inhibitor or angiotensin receptor blocker prior to study entry are eligible for randomization provided the last dose was taken at least 12 hours prior to receiving study medication.

All other medications approved for use are acceptable. The investigator should follow local guidelines for the administration of medications in combination with ACE inhibitors and angiotensin receptor blockers.

The patients must be instructed to inform the investigator of all concomitant medications, including those available over-the-counter. This information must be recorded in the patient's chart (source documents). Cardiovascular, antithrombotic, and antidiabetic medications, lipid-modulating agents, hormone replacement therapy, contraceptive agents, and non-steroidal antiinflammatory medications will be recorded by drug class in the CRF. In the very rare situations where open-label ACE inhibitors or angiotensin receptor blockers are deemed necessary, start/end dates will be required. Otherwise, reasons for administration, doses, and start/end dates will not be recorded.

3.4.5. Treatment Compliance

Records of study medication dispensed and returned, dosages administered, and intervals between visits will be kept by the site during the study. Drug accountability will be checked by the field monitor during site visits and at the completion of the trial by review of the study medication records and by checking the tear-off part of the study drug label attached to the CRF.

3.5. Visit Schedule and Assessments

3.5.1. Visit Schedule

The day of randomization will be considered Day 1.

A patient can be randomized on the day of myocardial infarction (must be at least 12 hours after the onset of symptoms) or on any day up to and including the tenth day after the onset of symptoms.

TABLE 3.5.1-1
Visit schedule

| | VISITS | | | | | | | | | | | | | | | |
|---|---|---|---|---|---|---|---|---|---|---|---|---|---|---|---|---|
| | | | | | | | | | | | | | | | 15 | 16 |
| | 1 | 2 | 3 | 4 | 5 | 6 | 7 | 8 | 9 | 10 | 11 | 12 | 13 | 14 | (1) | (1) |
| | Day 1 | Month (±15 days) | | | | | | Month (±20 days) | | | | | | | | |
| RANDOMIZATION | 1 | 0.5 | 1 | 3 | 6 | 9 | 12 | 16 | 20 | 24 | 28 | 32 | 36 | 40 | 44 | 48 |
| ON DAY 1: | | (2) | | | | | | | | | | | | | | |
| 12 hours to 10 days | | | | | | | | | | | | | | | | |
| after onset of an | | | | | | | | | | | | | | | | |
| acute myocardial | | | | | | | | | | | | | | | | |
| infarction | | | | | | | | | | | | | | | | |
| Informed Consent | X | | | | | | | | | | | | | | | |
| Demographics | X | | | | | | | | | | | | | | | |
| Medical history | X | | | | | | | | | | | | | | | |
| 12-lead ECG | X | | | | | | | | | | | | | | | |
| assessment (3) | | | | | | | | | | | | | | | | |
| Cardiac enzymes | X | | | | | | | | | | | | | | | |
| (3) | | | | | | | | | | | | | | | | |
| Chest X-ray (3) | X | | | | | | | | | | | | | | | |
| Evaluation of left | X | | | | | | | | | | | | | | | |
| ventricular systolic | | | | | | | | | | | | | | | | |
| dysfunction (3) | | | | | | | | | | | | | | | | |
| Evaluation of | X | | | | | | | | | | | | | | | |
| Inclusion/ | | | | | | | | | | | | | | | | |
| Exclusion criteria | | | | | | | | | | | | | | | | |
| Record Killip Class | X | | | | | | | | | | | | | | | |
| Vital signs (blood | X | X | X | X | X | X | X | X | X | X | X | X | X | X | X | X |
| pressure and heart | | | | | | | | | | | | | | | | |
| rate) and NYHA | | | | | | | | | | | | | | | | |
| functional class | | | | | | | | | | | | | | | | |
| Evaluation of | | X | X | X | X | X | X | X | X | X | X | X | X | X | X | X |
| endpoint criteria | | | | | | | | | | | | | | | | |
| Adverse events | X | X | X | X | X | X | X | X | X | X | X | X | X | X | X | X |
| Check titration | | X | X | X | X | X | X | X | X | X | X | X | X | X | X | X |
| Medication | X | X | X | X | X | X | X | X | X | X | X | X | X | X | X | |
| dispensed | | | | | | | | | | | | | | | | |
| Medication returned | | X | X | X | X | X | X | X | X | X | X | X | X | X | X | X |
| Serum creatinine | X | * | * | * | * | * | * | * | * | * | * | * | * | * | * | X |
| (local laboratory) | | | | | | | | | | | | | | | | (4) |
| Selected co- | X | X | X | X | X | X | X | X | X | X | X | X | X | X | X | X |
| medication: | | | | | | | | | | | | | | | | |
| Quality of life | | X | | | X | | X | | X | X | | | X | | | X |
| questionnaire | | | | | | | | | | | | | | | | |
| (EuroQol © ) (5) | | | | | | | | | | | | | | | | |
| Pharmacoeconomic | | X | X | X | X | X | X | X | X | X | X | X | X | X | X | X |
| assessment | | | | | | | | | | | | | | | | |
| Study completion | | | | | | | | | | | | | | | | X |
| sheet | | | | | | | | | | | | | | | | (4) |

(1) If the study duration is longer or shorter than the four years presented as an example in this table, the Visit 15 schedule may be repeated every four months until the study is completed or deleted as required. At the final study visit, the schedule presented for Visit 16 will be followed.
(2) Visit at Day 15 or at hospital discharge, whichever is sooner.
(3) One or more of these tests (performed prior to randomization as part of the patient's standard clinical evaluation and care) are needed to qualify the patient for the study.
(4) At the end of the study or premature treatment discontinuation
(5) The quality of life questionnaire will be required for only a subset of patients.
* At the investigator's discretion. Only required prior to the initial titration to Steps II, III, or IV. Results of laboratories performed as part of the patient's standard clinical evaluation and care should be used to evaluate potential study endpoints and adverse events.

Visit Procedures

The study consists of two phases, 1) a study medication initiation and titration phase and 2) a maintenance phase. The duration of these two phases depends upon the patient's status and response to study medication.

Randomization and initiation of study medication will occur at Visit 1 on Day 1. For most patients, this visit will occur in hospital.

Dose titration and maintenance will occur at Visits 2-16.

Visit 2 will occur on Day 15 or at hospital discharge, whichever is first. For patients not in hospital at the time of randomization, Visit 2 will occur on Day 15.

Visits 3-16 are planned as out-patient visits, but depending on the patient's status, may occur in hospital. They are to be performed at specified time points but some flexibility is allowed. During the first year, the visit may take place up to 15 days before or after the protocol-defined date. During subsequent years, the visit may take place up to 20 days before or after the protocol-scheduled visit.

Note: One month is a calendar month, e.g., July 15 to August 15

When used in pregnancy during the second and third trimesters, drugs that act directly on the renin-angiontensin system can cause injury and even death to the developing fetus. Pre-menopausal women who are using acceptable methods of birth control (See inclusion criteria.) and who are not surgically sterile should be checked periodically during the study to rule out pregnancy. If a pregnancy is detected, study medication should be discontinued and the Coordinating Center notified immediately.

Visit 1 (Day 1, Randomization and Initiation of Study Medication)

Before Randomization

Evaluate patient history and current status according to the study inclusion and exclusion criteria.

If the patient is eligible for randomization:
Obtain written informed consent.
Record demographic data, medical history and concomitant medications by drug class (cardiovascular, antithrombotic, and antidiabetic medications, lipid-modulating agents, hormone replacement therapy, contraceptive medications/devices, antidepressants, and non steroidal antiinflammatory medications).
Record the highest Killip Class prior to randomization
Record the heart rate, blood pressure and NYHA functional class.
Record the baseline serum creatinine measurement (local lab).
Randomize the patient.
   Randomization

Randomize the patient via the 24-hour telephone call-in system (Q-tone). Record in the CRF the site number, the patient number, and the date and time of randomization. This date is to be considered Day 1 and is the reference for planning subsequent visits.

Randomization should occur as soon as possible but no earlier than 12 hours and no later than the end of the 10th day after the onset of symptoms of myocardial infarction.

After Randomization

The first dose of study medication is to be given to the patient as soon as possible after randomization. If for any reason, a temporary contra-indication to study medication is anticipated, randomization should be postponed accordingly.

Give the first dose of titration Step I and monitor the patient closely. Do not titrate study medication to titration Step II before the morning of Day 2.

Complete the Serious Adverse Event CRF for any serious adverse events that occur after obtaining informed consent and are suspected to be related to the administration of study medication. (See Section 3.5.3: Safety assessments, for the definitions to be used in evaluating the seriousness of an adverse event and for determining the relationship of an adverse event to study medication.)

Visit 2 (15 Days After Randomization or at Hospital Discharge, Whichever Comes First)

Record heart rate, blood pressure, and NHYA functional class.

From Day 2 onwards, continue with the titration schedule as described in Section 3.4.1: Investigational therapy and reference therapy. Up-titration can be carried out at anytime during the day (morning, midday, or evening dose). Record each titration step since the last visit.

Record serum creatinine (only required for the initial titration to Steps II, III and IV).

For adverse events occurring since randomization:
Complete the Serious Adverse Event CRF for any serious adverse events that are suspected to be related to the administration of study medication. (See Section 3.5.3: Safety assessments, for the definitions to be used in evaluating the seriousness of an adverse event and for determining the relationship of an adverse event to study medication.)
Record serious events not suspected to be related to study medication in the CRF and/or endpoint documentation.
Record the occurrence of any pre-defined safety and tolerability parameters in the CRF. (See Section 3.5.3: Safety assessments.)
Record any non-serious adverse events in the patients study chart (source documents).
Assess and record potential efficacy and safety endpoints since the time of randomization.
Count returned study medication and complete study medication log.
Dispense new study medication and complete study medication log.
Record concomitant medication drug classes.
Have the patient complete the quality of life questionnaire (only required for the quality of life subset of patients).
Complete the pharmacoeconomic assessment.
   Visit 3 (30 Days After Randomization)

Record heart rate, blood pressure, and NYHA functional class.

Continue with the titration schedule as presented in Section 3.4.1: Investigational therapy and reference therapy. Up-titration can be carried out at anytime during the day (morning, midday, or evening dose). Record each titration step since the last visit.

Record serum creatinine (only required for the initial titration to Steps II, III and IV).

For adverse events occurring since the last visit:
Complete the Serious Adverse Event CRF for any serious adverse events that are suspected to be related to the administration of study medication. (See Section 3.5.3: Safety assessments, for the definitions to be used in evaluating the seriousness of an adverse event and for determining the relationship of an adverse event to study medication.)
Record serious events not suspected to be related to study medication in the CRF and/or endpoint documentation.
Record the occurrence of any pre-defined safety and tolerability parameters in the CRF. (See Section 3.5.3: Safety assessments.)
Record any non-serious adverse events in the patients study chart (source documents).
Assess and record potential efficacy and safety endpoints since the last visit.
Count returned study medication and complete study medication log.
Dispense new study medication and complete study medication log.
Record concomitant medication drug classes.
Complete the pharmacoeconomic assessment.
   Visits 4 to 15 (Visits 4, 5, 6, and 7 will Occur at 3, 6, 9, and 12 Months After Randomization. Subsequent Visits Will Occur Every Four Months Until Study Completion.)

Record heart rate, blood pressure, and NYHA functional class.

Continue with the titration schedule as presented in Section 3.4.1: Investigational therapy and reference therapy. Up-titration can be carried out at anytime during the day (morning, midday, or evening dose). Record each titration step since the last visit.

Record serum creatinine (only required for the initial titration to Steps II, III and IV).

For adverse events occurring since the last visit:
Complete the Serious Adverse Event CRF for any serious adverse events that are suspected to be related to the administration of study medication. (See Section 3.5.3: Safety assessments, for the definitions to be used in evaluating the seriousness of an adverse event and for determining the relationship of an adverse event to study medication.)
Record serious events not suspected to be related to study medication in the CRF and/or endpoint documentation.
Record the occurrence of any pre-defined safety and tolerability parameters in the CRF. (See Section 3.5.3: Safety assessments.)
Record any non-serious adverse events in the patient's study chart (source documents).
Assess and record potential efficacy and safety endpoints since the last visit.
Count returned study medication and complete study medication log.
Dispense new study medication and complete study medication log.
Record concomitant medication drug classes.
Have the patient complete the quality of life questionnaire at Visits 5, 7, 9, 10, and yearly thereafter (only required for the quality of life subset of patients).
Complete the pharmacoeconomic assessment.
   Visit 16 (Final Visit, Month 48 or at Study End)

Record heart rate, blood pressure, and NYHA functional class.

Record serum creatinine.

For adverse events occurring since the last visit:
Complete the Serious Adverse Event CRF for any serious adverse events that are suspected to be related to the administration of study medication. (See Section 3.5.3: Safety assessments, for the definitions to be used in evaluating the seriousness of an adverse event and for determining the relationship of an adverse event to study medication.)
Record serious events not suspected to be related to study medication in the CRF and/or endpoint documentation.
Record the occurrence of any pre-defined safety and tolerability parameters in the CRF. (See Section 3.5.3: Safety assessments.)
Record any non-serious adverse events in the patient's study chart (source documents).
Assess and record potential efficacy and safety endpoints since the last visit.
Count returned study medication and complete study medication log.
Dispense no further study medication to the patient.
Record concomitant medication drug classes.
Have the patient complete the quality of life questionnaire (only required for the quality of life subset of patients).
Complete the pharmacoeconomic assessment.
Complete the Study Completion Sheet of the CRF.

Patients who are permanently discontinued from double-blind study medication for any reason must, if at all possible, complete the protocol-specified visits until the end of the study or until death. Such patients will not be dispensed study medication at the visits following treatment discontinuation. If for documented reason, the patient cannot come for follow-up visits, telephone follow-up is permitted. The investigator must aim to obtain as complete follow-up as possible in all patients including, at the very least, the patient's vital status. The study will end when the required number of primary endpoints has been reached. This may occur prior to or after Month 48. If the study ends prior to Month 48, the procedures listed for Visit 16 will be completed for all patients. If the study is extended beyond Month 48, the procedures listed for Visit 15 will be completed every 4 months until study end at which point the procedures listed for Visit 16 will be completed.

3.5.2. Efficacy Assessments

Documentation for occurrences of potential primary or secondary efficacy endpoints will be required for submission to the Endpoint Committee. The Endpoint Committee will adjudicate causes of death and selected secondary endpoints based upon pre-defined definitions and procedures for this study. The process of endpoint adjudication, and the definitions and required documentation for the primary and secondary endpoints are included in the Endpoint Manual.

Primary Efficacy Parameters

The primary efficacy parameter is all-cause mortality (time to death).

Secondary Efficacy Parameters

Secondary efficacy parameters are as follows:
All-cause (unplanned and elective) hospitalization
All-cause mortality and all-cause hospitalization
Hospitalization for heart failure (defined as unplanned intravenous treatment of new or worsening heart failure with inotropic agents, diuretics, or vasodilators requiring or occurring during any hospital admission or overnight stay in a health care facility)
All-cause mortality and hospitalization for heart failure
Cardiovascular mortality (defined as sudden death, or death attributed to recurrent myocardial infarction, heart failure, a cardiovascular procedure, stroke, or other cardiovascular etiology)
Cardiovascular mortality and hospitalization for heart failure
Cardiovascular mortality, hospitalization for heart failure, and recurrent non-fatal myocardial infarction
Cardiovascular mortality, hospitalization for heart failure, recurrent non-fatal myocardial infarction, and coronary revascularization procedures (defined as unplanned and elective percutaneous coronary angioplasty, stent, other percutaneous coronary revascularization, and coronary artery bypass surgery)
Cardiovascular morbidity (defined as hospitalization for heart failure, unplanned hospitalization for non fatal recurrent myocardial infarction, unstable angina, sudden cardiac arrest with resuscitation, stroke, transient ischemic attack, other cardiovascular-related unplanned hospitalization)
All cause mortality and cardiovascular morbidity
Cardiovascular mortality and cardiovascular morbidity
Sudden death and sudden cardiac arrest with resuscitation
Fatal and non-fatal recurrent myocardial infarction
Coronary revascularization procedures
Cardiovascular procedures (defined as coronary revascularization procedures, cardiovascular procedures for congestive heart failure, heart transplant, or other vascular procedures)
All cause mortality at 30 days.
   3.5.3.5Safety Assessments

Safety assessments will consist of monitoring and recording the pre-defined safety and tolerability endpoints (see below), all serious adverse events, and the regular measurement of vital signs.

Results of all safety assessments (e.g., physical examinations or laboratories) performed as part of the standard evaluation and care of the patient should be maintained in the patient's study chart (source documents).

Pre-Defined Safety and Tolerability Parameters

The following pre-defined safety and tolerability endpoints are known side effects of either captopril and/or valsartan. Information on the occurrence of these adverse events will be collected and recorded on the CRF for all patients.

Symptomatic Hypotension

Symptomatic hypotension is defined as one of the following: hypotension (including first-dose hypotension) accompanied by symptoms (e.g., dizziness, faintness, diaphoresis), or persistent hypotension leading to dose reduction or temporary interruption or permanent discontinuation of study medication. This symptom is not considered a reason for the investigator to unblind study medication. However, the DSMB will be reviewing the rates of occurrence of these events and may unblind if deemed necessary.

Renal Dysfunction

Renal dysfunction is defined as one of the following: death from renal failure, end-stage renal disease requiring chronic dialysis or renal transplant, or an increase in serum creatinine leading to temporary interruption or permanent discontinuation of study medication. This symptom is not considered a reason for the investigator to unblind study medication. However, the DSMB will be reviewing the rates of occurrence of these events and may unblind if deemed necessary.

Dry Cough

A dry cough is characteristically dry, persistent, and occasionally paroxysmal. When related to inhibition of the angiotensin system, it usually develops between 1 week and 6 months after initiation of therapy. It is not a cough with production of sputum or a dry cough with cause that can be identified, such as viral bronchitis or pulmonary congestion. This symptom is not considered a reason for the investigator to unbind study medication. However, the DSMB will be reviewing the rates of occurrence of these events and may unblind if deemed necessary.

Angioedema

Angioedema is characterized by a rapid swelling in the nose, throat, mouth, glottis, larynx, lips, and/or tongue. When related to inhibition of the angiotensin system, this rare event is apparently not dose-related and usually develops within the first week of therapy, usually within the first few hours after the initial dose. Airway obstruction and respiratory distress may lead to death. Study treatment must be permanently discontinued. Unblinding of study medication could be considered by the investigator. The DSMB will be reviewing the rates of occurrence of these events and may unblind if deemed necessary.

Once ACE inhibitors or angiotensin receptor blockers are stopped, angioedema usually disappears within hours; meanwhile, the patient's airways should be protected, and if necessary, epinephrine, or an antihistamine, and/or corticosteroid should be administered.

Adverse Events

Adverse events will be recorded in the CRF or the Serious Adverse Event (SAE) form if they meet the following criteria:
Primary and secondary efficacy parameters (as described in Section 3.5.2)
Pre-specified safety and tolerability parameters (known side effects of either captopril and/or valsartan) as described in the previous section
Serious adverse events (as described in the following section).

Other non-serious adverse events will not be collected in the CRF. However, information about all adverse events, whether volunteered by the patients, discovered by investigator questioning, or detected through physical examination, laboratory test or other means, will be recorded in the patient's study chart (source documents) and the events will be followed and treated as appropriate. An adverse event is any undesirable sign, symptom or medical condition occurring after starting study treatment, even if the event is not considered to be treatment-related. Medical conditions/diseases present before starting study treatment are considered adverse events only if they worsen after starting study treatment. Abnormal laboratory values or test results constitute adverse events only if they induce clinical signs or symptoms or require therapy or a change in therapy.

Serious Adverse Events (SAEs)

A serious adverse event is defined in general as an untoward (unfavorable) event which:
1. is fatal or life-threatening,
2. required or prolonged hospitalization,
3. was significantly or permanently disabling or incapacitating,
4. constitutes a congenital anomaly or a birth defect,
5. is medically significant (may jeopardize the subject and may require medical or surgical intervention to prevent one of the outcomes listed above).

Events not considered to be serious adverse events are hospitalizations occurring under the following circumstances: were planned before entry into the clinical study; occur on an emergency, outpatient basis and do not result in admission (unless fulfilling the seriousness criteria above); are part of the normal treatment or monitoring of the studied indication and are not associated with any deterioration in condition.

The relationship between the administration of study drug and the occurrence of the adverse event is described as belonging to one of only 2 categories, either suspected by the investigator or not suspected by the investigator.

Relationship of Adverse Events to Study Drug

| | |
|---|---|
| Not | The temporal relationship of the clinical event to study drug |
| suspected | administration makes a causal relationship unlikely, |
| | or other drugs, therapeutic interventions or underlying |
| | conditions provide a sufficient explanation for the |
| | observed event. |
| Suspected | The temporal relationship of the clinical event to |
| | study drug administration makes a causal relationship |
| | possible, and other drugs, therapeutic interventions |
| | or underlying conditions do not provide a sufficient |
| | explanation for the observed event. |

To ensure patient safety each serious adverse event suspected by the investigator to be related to study medication must be reported to the study Coordinating Center within 24 hours of learning of its occurrence.

Serious adverse events not suspected by the investigator to be related to study medication will be reported to the Coordinating center with the CRF and/or endpoint documentation.).

For detailed instructions about completing and returning Serious Adverse Event Report Forms to the study Coordinating Center refer to Section 9.1.1: Instructions for rapid notification of serious adverse events.

Laboratory Evaluations

Serum creatinine will be performed at Visit 1, at the end of the study or at the time of or permanent discontinuation of study medication, and prior to the initial up-titration of the study medication to Steps II, III and IV.

Other than serum creatinine, no laboratory measurements are required. Laboratory measurements should be performed as required for the usual care of the patient and where possible the results should be included in the patient's study chart (source documents). If a particular laboratory value is needed to enable the assessment of a potential endpoint, that value should be included in the patient's study records for submission to the Endpoint Committee.

Each participating center will use its local laboratory for laboratory evaluations. A central laboratory will NOT be employed. The normal ranges of the local laboratory serve as the reference for the patients of the particular center. If in the course of the study, a patient is hospitalized in a non-participating center, the local lab and normal ranges of that hospital will be considered for that hospitalization.

Vital Signs

The highest Killip Class prior to randomization will be recorded at Visit 1.

Heart rate and blood pressure, will be measured at each visit. Blood pressure is to be measured before any upward titration (See Section 3.4.1: Investigational therapy and reference therapy.) and to monitor treatment tolerability.

New York Heart Association (NYHA) functional class will be recorded at each visit.

Special Tests

Cardiac enzymes and the results of a 12-lead ECG, chest X-ray, echocardiogram, radionuclide ventriculogram, or ventricular contrast angiogram may be needed to confirm a patient's eligibility for the study. Results of these tests, performed when needed as part of the patient's standard clinical evaluation and care, should be included in the patient's study chart (source documents). If a particular test result is needed to enable the assessment of a potential endpoint, that value should be included in the patient's study records for submission to the Endpoint Committee.

3.5.4. Drug Levels and Pharmacokinetic Assessments

No drug levels or pharmacokinetic assessments are planned.

3.5.5. Resource Utilization and Quality of Life Assessments

The resource utilization parameters to be followed during the study include:
In-patient hospitalizations
Outpatient visits to health care providers
Outpatient cardiovascular procedures.

The quality of life assessment will utilize the EuroQol© instrument (21-23). This two-part instrument consists of a six-item functional status assessment and a thermometer visual analogue scale. The EuroQol© is self-administered by patients. The quality of life assessment will be conducted in a subset of the randomized patients.

4. Protocol Amendments, Other Changes in Study Conduct

4.1. Protocol Amendments

Changes to the protocol (except for minor administrative changes) will be made in the form of an amendment. Based upon their review of the interim study data, the DSMB will have the authority to recommend amendments to the protocol. The Executive Committee will review and approve all protocol amendments. Prior to implementation, all amendments will be reviewed and approved by the local health authorities and ethical review boards as required (See Section 9.2.1: Changes to the protocol.).

4.2. Other Changes in Study Conduct

Changes in study conduct are not permitted. Any unforeseen changes in study conduct will be recorded in the clinical study report.

5. Data Management

5.1. Data Collection

Investigators must enter the information required by the protocol onto the Case Report Forms (CRFs). Field monitors will review the CRFs for completeness and accuracy, and instruct site personnel to make any required corrections or additions.

The CRFs will be forwarded to the study data management centers. One copy of the CRF will be retained at the investigational site. Once the CRFs are received by the data management centers, their receipt will be recorded, and they will be forwarded to the responsible data management staff for processing.

Documentation supporting the primary and secondary endpoints will be forwarded to the data management centers for adjudication by the Endpoint Committee. The required documentation is outlined in the Endpoint Manual.

5.2. Database Management and Quality Control

Database management and quality control for this study are the responsibility of Duke Clinical Research Institute, Durham, N.C., USA.

Data items from the CRFs will be entered into the study database using double data entry with verification upon second entry. Text items (e.g., comments) will be entered once and checked manually against the CRFs.

Subsequently, the information entered into the database will be systematically checked by data management staff, using error messages generated from validation programs and database listings. Obvious errors will be corrected by data management center personnel. Other errors, omissions or questions will be entered on data query forms, which will be returned to the investigational site for resolution. After the investigator response is received at the data management center, the resolutions will be entered into the database. A copy of the signed data query form will be kept with the CRFs. Quality control audits of all key safety and efficacy data in the database will be made at designated times during the study.

Coexistent diseases and adverse events will be coded using a standard coding dictionary, MEDDRA. Concomitant medications will be coded using a standard medication dictionary, WHO DRL.

When the database has been declared to be complete and accurate, the database will be locked and unblinded. Any changes to the database after that time can only be made by joint written agreement between the Clinical Trial Leader, the Trial Statistician and the Data Manager.

6. Statistical Methods

6.1. Statistical Methods to be Employed

The primary hypotheses to be investigated are whether valsartan is either superior to captopril (“superiority”) or as effective as captopril (“non-inferiority”), and whether the combination of captopril and valsartan is superior to captopril as monotherapy. The primary efficacy variable for these comparisons is time to death, and the hypotheses will be tested using a Cox regression analysis (details are contained in Section 6.1.5.). Secondary efficacy variables will also be tested using Cox regression analyses.

The data will be analyzed by Novartis. Any data analyses carried out independently by the investigators should be submitted to Novartis before publication or presentation.

The data from all centers that participate in this protocol will be combined, so that an adequate number of patients are available for analysis.

Data will be summarized with respect to demographic and baseline characteristics, efficacy observations and measurements, and safety observations and measurements.

6.1.1. Populations

Primary Analysis Population:

The primary analysis population will consist of all randomized patients who receive trial medication. In analyses based on this population, all events that occur up to and including the time of trial completion will be included in analyses, regardless of whether the events occur before or after permanent discontinuation of double-blind treatment.

Per-Protocol Population:

The per-protocol population will consist of all patients who satisfy the protocol inclusion criteria regarding having sustained an acute myocardial infarction (see Section 3.3.2), and who receive, at least once, titration Step II of study medication (see Section 3.5.).

In per-protocol time-to-event analyses, if a patient permanently discontinues double-blind treatment and the event has not occurred by the date of permanent discontinuation indicated on the case report form, then the time-to-event for that patient will be considered censored as of that date, regardless of the reason for discontinuation. Thus, events occurring prior to permanent discontinuation will be included in per-protocol analyses as non-censored events, and events occurring subsequent to discontinuation will not be included.

For a patient who temporarily discontinues from double-blind treatment and for whom that discontinuation is continuous for two consecutive visits, events occurring prior to the second consecutive visit will be included in the per-protocol analyses as non-censored events, and events occurring subsequent to the second consecutive visit will not be included.

In addition, for patients who have not permanently discontinued trial treatment, a patient will be considered censored at any point of the trial at which it is indicated on the case report forms at two consecutive visits that the patient has received an ACE inhibitor or angiotensin receptor blocker other than study medication (the censoring will be considered to have occurred at the date of the second of the two consecutive visits).

Populations for the Primary and Secondary Analyses:

The primary efficacy variable will be analyzed using the primary analysis population for the superiority and non-inferiority comparisons of captopril versus valsartan, and for the superiority comparison of the combination versus captopril. Each of these comparisons will also be performed using the per-protocol population and the set of all randomized patients, in order to assess the sensitivity of the conclusions obtained from the analyses using the primary analysis population. Other sensitivity analyses for the primary variable may also be considered as needed.

All secondary variables will be analyzed using the primary analysis population.

Cardiovascular mortality (as defined in Section 3.5.2.) will also be analyzed using the per-protocol population.

Data Sets for the Interim Analysis:

Formal comparisons of the treatment arms, performed according to the interim analysis plan (see Section 6.1.7.), will be based on the primary analysis population and will include patients randomized prior to a cutoff date defined for each interim analysis. Other analyses, possibly using different populations, will be defined with input from the independent DSMB and documented in the DSMB Manual, to be issued prior to the first analyses of any interim data.

6.1.2. Background and Demographic Characteristics

Appropriate summary statistics will be provided for the primary analysis population by treatment group, and by treatment group and country, for demographic and medical history characteristics, and for Killip class, blood pressure, and heart rate measured at Visit 1. P-values from comparisons of the treatment groups with respect to these variables will also be provided (these p-values are provided for descriptive purposes, and are not to be considered to define any formal basis for determining factors that should be included in statistical analysis models).

6.1.3. Study Medication

Summary statistics for duration of exposure to trial medication will be calculated by treatment group, and, if appropriate, by treatment group and dose level.

6.1.4. Concomitant Therapy

Summary statistics will be provided as appropriate. No formal analyses are planned.

6.1.5. Efficacy Evaluation

Primary Efficacy Variables

The primary efficacy variable is time to death. This will be calculated for each non-surviving patient as the difference between the date of death and the date of randomization.

Adjustment for Multiple Comparisons:

The primary goal of the trial will be achieved if valsartan monotherapy is found to be superior to, or as effective as, captopril, or if the combination of valsartan and captopril is found to be superior to captopril. In order to maintain a global significance level ≦0.05 for these tests, overall significance levels of 0.0253 (Sidak adjustment) will be used; for the superiority hypotheses two-sided tests will be performed, and a one-sided test will be performed for the non-inferiority hypothesis. Note that testing for both superiority and non-inferiority of valsartan monotherapy versus captopril does not require further significance level adjustment, based on use of a closed test procedure (24).

Comparison of Captopril Versus Valsartan:

For the primary comparison between captopril and valsartan, both a superiority hypothesis and a non-inferiority hypothesis will be formally investigated.

For the superiority comparison, the null hypothesis is that the risk ratio (hazard ratio for mortality) between captopril and valsartan is equal to 1, versus the alternative hypothesis that it is not equal to 1:
H₀:λ₂/λ₁=1 against H₁:λ₂/λ₁≠1
where λ₁ and λ₂ are the hazard rates for captopril and valsartan, respectively.

For testing whether valsartan is at least as effective as captopril, the null hypothesis is that the risk ratio between captopril and valsartan is at least 1+Δ, versus the alternative hypothesis that it is less than 1+Δ:
H₀:λ₂/λ₁≧1+Δ against H₁:λ₂/λ₁<1+Δ
where Δ is the acceptance range within which the two treatments are considered to be equivalent, and is defined to be 0.13. This value has been selected based on a meta-analysis of the AIRE, TRACE, and SAVE studies (4, 8-10), which indicated an estimated 22.5% hazard ratio benefit for an ACE inhibitor relative to placebo, with a 95% confidence interval of 14.4% to 29.8% (see Section 3.2). Thus, using Δ=0.13 ensures that if the test criterion is achieved, valsartan will have demonstrated significant benefit versus placebo, even in a worst case, and would demonstrate that nearly half of the estimated benefit of an ACE inhibitor has been preserved. It can further be estimated that the least efficacious observed outcome for valsartan which would achieve this criterion would be one not more than 3% worse than captopril. Thus, in order to claim valsartan is as effective as captopril, either the estimated hazard for valsartan will be less than that of captopril, or not more than about 3% higher than that of captopril (corresponding, for example, to observing total mortality rates during the trial of 20% for captopril and 20.6% for valsartan).
Comparison of the Combination of Captopril and Valsartan Versus Captopril:

For the primary comparison between the combination of captopril and valsartan and captopril monotherapy, a superiority test will be performed. The null hypothesis is that the risk ratio (hazard ratio) between the combination therapy and captopril is equal to 1, versus the two-sided alternative that the risk ratio is not equal to 1:
H₀:λ₃/λ₁=1 against H₁:λ₃/λ₁≠1
where λ₁ and λ₃ are the hazard rates for captopril and the combination, respectively.

For the secondary objective involving the comparison of the combination to valsartan, the hypotheses are defined analogously.

Statistical Model:

For comparisons involving the primary variable, as well as for other time-to-event variables, analyses will be performed using Cox regression models. The primary analysis model for each comparison will contain treatment group, age (as a continuous covariate), and occurrence of a previous myocardial infarction. The assumption of proportionality of the treatment arm hazard functions (i.e., constant hazard ratio) will be investigated, and implications for the primary analysis results of any non-proportionality will be considered. Supplemental logrank tests will also be performed. Exploratory analyses will be performed to address the impact of other potentially important prognostic factors.

Criteria for Efficacy:

Valsartan monotherapy will be considered superior to captopril monotherapy if the difference between these treatment arms, using the primary analysis population and the Cox regression analysis of the primary variable, is statistically significant in favor of valsartan using a two-sided level of 2.53%.

If valsartan is not shown to be superior to captopril, it will be concluded that valsartan is at least as effective as captopril if the upper limit of the confidence interval for the hazard ratio (derived from the Cox regression estimate and using a one-sided significance level of 2.53%) is less than 1.13.

The combination of captopril and valsartan will be considered superior to captopril if the difference between these treatment arms, using the primary analysis population and the Cox regression analysis of the primary variable, is statistically significant in favor of the combination using a two-sided significance level of 2.53%.

Exploratory Subgroup Analyses

For the primary variable and for the composite death, reinfarction, hospitalization for heart failure, descriptive summaries will be presented and exploratory analyses will be considered as appropriate to investigate the possibility of differential treatment effects in subgroups defined by the following factors: age, gender, race, prior MI, history of hypertension, diabetes, hyperlipidemia or smoking, time to randomization, Killip class, infarct location and type, coronary revascularization procedures prior to and at the time of the index myocardial infarction, evidence of LV dysfunction or heart failure, and the use of beta blockers, aspirin, ACE inhibitors or ARBs, or thrombolytics prior to randomization.

Secondary Efficacy Variables

Secondary efficacy variables are defined in Section 3.5.2. For all composite endpoints the outcome variable is defined as the occurrence of at least one component of the composite, regardless of whether or not more than one component may have occurred during the course of the trial; thus, each patient is counted once in the analysis.

Analysis of Secondary Efficacy Variables

Secondary variable analyses will be based upon the primary analysis population; cardiovascular mortality will additionally be analyzed using the per-protocol population. Each secondary efficacy variable will be analyzed using the same Cox regression model defined for the primary variable. Additional follow-up analyses, possibly addressing multiple occurrences of events per patient, will be considered as appropriate.

Summary Statistics and Frequency Distributions for the Primary and Secondary Efficacy Variables:

For all primary and secondary efficacy variables, the percentage of patients with the event occurring until trial completion and the percentage of events that occur during the double blind treatment period will be presented by treatment group. The total mortality rate by treatment group will also be presented for each level of the variables defining key subgroups, as described above.

For time-to-event variables, plots of the Kaplan-Meier survival probabilities by treatment group will be provided.

6.1.6.5Safety Evaluation

The assessment of safety is based mainly on the frequency of the pre-defined safety and tolerability parameters and serious adverse events suspected by the investigator to be related to study medication. Other safety data (e.g., vital signs) will be considered and summarized as appropriate.

Serious adverse events suspected by the investigator to be related to study medication will be summarized for each treatment group by presenting the number and percentage of patients having any serious related adverse event, having a serious related event in each body system and having each individual serious related adverse event.

6.1.7. Interim Analyses

Two formal interim analyses for the primary efficacy endpoint will be performed. Cutoff dates for the first and second interim analyses will be approximately equally spaced with respect to the targeted total number of deaths prior to study completion. The interim analyses are thus planned to be performed to coincide with the DSMB meetings closest to the times when 900 and 1800 deaths have been reported. For each interim analysis the data set analyzed will consist of all patients in the primary analysis population randomized prior to the cutoff date.

O'Brien-Fleming-type boundaries with a Lan-DeMets alpha spending function (25) will be used to determine significance criteria. A cumulative two-sided significance level of 2.53% will be used to indicate formal statistical significance for each of the three pairwise comparisons of the treatment arms. Because information on mortality will be provided to the independent DSMB for each of the planned twice-yearly safety reviews, the O'Brien-Fleming boundary criteria for the interim and final analyses will adjust for these safety analyses as well. The trial may be stopped early, or a treatment arm may be discontinued, if a significant difference between groups is indicated by crossing a pre-specified boundary at an interim analysis.

Conditional probability calculations, estimating the probabilities that a significant difference between each pair of treatment arms will be achieved, will also be calculated along with the formal efficacy analyses as an additional guidance for decision making by the DSMB. These will allow the DSMB to consider criteria less stringent than the formal boundaries if there is a strong tendency towards a benefit for captopril over either of the other treatment arms. No criteria are defined to establish non-inferiority of valsartan relative to captopril based on an interim analysis.

The interim analyses will be performed outside Novartis by an independent statistical center, and the results will be reviewed by the independent DSMB. Investigators, Novartis employees and others who are involved in the conduct of the trial and in the analysis of the final trial results, or who have contact with study centers, will remain blinded to the treatment codes and to the interim analysis results until all monitoring decisions have been made and the database has been locked for final analysis.

The trial may be stopped early, or a treatment arm may be discontinued, if a significant difference between groups is indicated by crossing a pre-specified boundary at an interim analysis. If the study is terminated early, final reporting and analysis will include all data (not just on the data available for the interim analysis on which the decision to terminate was based).

6.1.8. Other Topics

Pharmacoeconomic Data

Information on hospitalizations, number of hospital days, number of outpatient cardiovascular procedures, and total hospital days will be assessed for each of the three treatment arms. Analysis will include descriptive statistics for each resource category.

Appropriate tests will be performed to determine whether utilization of resources differ between treatment groups. Analysis of resource use will be documented separately from the clinical study report.

Quality of Life Data

Quality of life assessment is an integral substudy of this protocol in specified countries. An analysis of EuroQol© scores across treatment groups will include descriptive statistics for each treatment arm. Comparison between treatment arms will be based on analysis of covariance (ANCOVA) using baseline EuroQol© scores as a covariate. The results of the quality of life analysis will be documented separately from the clinical study report.

6.2. Sample Size and Power Considerations

In sample size calculations, an annual mortality rate of 6.9% for captopril patients is assumed; this is based on results in the AIRE, SAVE, and TRACE studies, and the use of a similar high-risk population (4, 8-10).

Power Consideration for the Superiority Hypotheses:

It is hypothesized that the benefit of the valsartan plus captopril combination over captopril monotherapy may be in the range of a 15%-17.5% reduction in the risk of death, i.e., λ₃=k λ₁, where k is between 0.825 and 0.85. Using a two-sided significance level of 0.0253, obtaining a total of 1700 primary events in these two treatment arms will provide 95.4% power for detecting a 17.5% reduction in mortality risk, and 85.9% power for detecting a reduction of 15% (these calculations reflect a 2% adjustment for the planned O'Brien-Fleming interim analysis scheme). The same power results apply for demonstration of superiority of valsartan over captopril under the same assumptions concerning the benefit of valsartan. Thus, requiring 1700 events in the valsartan and captopril monotherapy treatment groups is considered to provide sufficient power to test the superiority hypotheses.

Power Consideration for the Non-Inferiority Hypothesis:

It is desired that the non-inferiority comparison have adequate power to demonstrate that valsartan is as effective as captopril if the true benefit for valsartan is in the range 0-2.5%. Using a one-sided significance level of 0.0253, a total of 1850 primary events in these two treatment arms will provide 88.1% power if valsartan is actually 2.5% better than captopril, and 74.0% power if the risk of mortality is identical in these two treatment groups.

Sample Size Determination

According to the information in the two preceding paragraphs, a total of
½(1700+1850+1850)=2700
events will provide adequate power to address the primary objectives (the power for superiority will be slightly larger than specified above because a slightly larger number of events is required for the non-inferiority hypothesis). Assuming a control group hazard rate corresponding to the 6.9% annual mortality rate referred to above, an 18-month enrollment period, a total trial duration of 48 months, and an inflation of approximately 7.5% to account for dropouts and the interim analysis plan, 14,500 patients (about 4833 per treatment arm) would be required to be enrolled.
Definition of Trial Completion

The trial is planned as a maximum information trial, i.e. the trial duration depends on a pre-specified number of 2700 patient deaths among all three treatment groups combined. The actual length in time of the trial will depend on the observed death rates, the patient accrual rate and length of the accrual period, and is expected to be about 4 years. In case the required number of events has not been observed after a trial duration of 6 years, the trial will be closed and considered completed.

7. Notable Laboratory Value Criteria, Special Methods and Scales

7.1. Criteria for Clinically Notable Laboratory Abnormalities

Except for serum creatinine as specified in the Visit Schedule (See Section 3.5.1.), the results of routine laboratory measurements will not be recorded in the CRF. Laboratory values obtained as part of the patient's standard care and evaluation may be needed to support a serious adverse event suspected to be related to study medication or the occurrence of a study endpoint and should be kept in the patient's study chart (source documents).

7.2. Special Methods and Scales

The EuroQol© instrument (21-23) will be used to assess Quality of Life.

### 8. REFERENCE LIST

Pfeffer, M A. ACE inhibitors in acute myocardial infarction. N Engl J Med 1995; 332: 118-120. ACE-inhibitor Myocardial Infarction Collaborative Group. Indications for ACE-inhibitors in the early treatment of acute myocardial infarction: systematic overview of individual data from 100,000 patients in randomized trials. Circulation 1998; 97: 2202-2212.
Pfeffer M A. ACE inhibitors in acute myocardial infarction. Patient selection and timing. [Editorial]. Circulation 1998; 97: 2192-2194.
Flather M D, Kober L, Pfeffer M A, Torp-Pedersen C, Hall A S, Murray G, Ball S G, Braunwald E, Yusuf S. Meta-analysis of individual patient data from trials of long-term ACE-inhibitor treatment after acute myocardial infarction (SAVE, AIRE, and TRACE Studies) (Abstract). Circulation 1997; 96: 1-706.
Ryan T J, Anderson J L, Antman E M et al. ACC/AHA guidelines for the management of patients with acute myocardial infarction. A report of the American College of Cardiology/American Heart Association Task Force on Practice Guidelines (Committee on Management of Acute Myocardial Infarction). J Am Coll Cardiol 1996; 28:1328-1428.
The task force on the management of acute myocardial infarction of the European Society of Cardiology. Acute myocardial infarction: pre-hospital and in-hospital management. Eur Heart J 1996; 17: 43-63.
Hennekens C H, Albert C M, Godfried S L, Gaziano J M, Buring J E. Adjunctive drug therapy of acute myocardial infarction—evidence from clinical trials. N Engl J Med 1996; 335:1660-1667.
Pfeffer M A, Braunwald E, Moy et al on Behalf of the SAVE Investigators. Effect of captopril on mortality and morbidity in patients with left ventricular dysfunction after myocardial infarction. Results of the Survival and Ventricular Enlargement Trial. N Engl J Med 1992; 327;669-677.
The Acute Infarction Ramipril Efficacy (AIRE) Study Investigators. Effect of ramipril on mortality and morbidity of survivors of acute myocardial infarction with clinical evidence of heart failure. Lancet 1993; 342; 821-828.
Kober L, Torp-Pedersen C, Carlsen J E et al for the Trandolapril Cardiac Evaluation (TRACE) Study Group. A clinical trial of the angiotensin-converting-enzyme inhibitor trandolapril in patients with left ventricular dysfunction after myocardial infarction. N Engl J Med 1995; 333; 1670-1676.
Heusch G, Rose J, Ehring T. Cardioprotection by ACE inhibitors in myocardial ischaemia/reperfusion. Drugs 1997; 54 Suppl. 5: 31-41.
Remme W J. Bradykinin-mediated cardiovascular protective actions of ACE inhibitors a new dimension in anti-ischaemic therapy? Drugs 1997; 54 Suppl. 5: 59-70.
Materson B I. Adverse effects of angiotensin-converting enzyme inhibitors in antihypertensive therapy with a focus on quinapril. Am J Cardiol 1992; 69:46C-53C.
Pitt B, Segal R, Martinez F A et al. Randomised trial of losartan versus captopril in patients over 65 with heart failure (Evaluation of Losartan in the Elderly Study, ELITE). Lancet 1997; 349: 747-752.
Pfeffer M A, Hennekens C H for the HEART Study Executive Committee. When a question has an answer: Rationale for our early termination of the HEART trial. Editorial. Am J Cardiol 1995; 75:1173-75.
Note for Guidance on Statistical Principles for Clinical Trials. CPMP/ICH/363/96. Step 4, consensus guideline, 5 Feb. 1998.
Garg R, Yusuf S for the Collaborative Group on ACE inhibitor trials. Overview of randomized trials of angiotensin-converting enzyme inhibitors on mortality and morbidity in patients with heart failure. J Am Med Assoc 1995; 273:1450-56.
ISIS-4 Collaborative Group. ISIS-4: a randomized factorial trial assessing early oral captopril, oral mononitrate, and intravenous magnesium sulphate in 58050 patients with suspected acute myocardial infarction. Lancet 1995; 345: 669-85.
Chinese Cardiac Study Collaborative Group. Oral captopril versus placebo among 13 634 patients with suspected acute myocardial infarction: interim report from the Chinese Cardiac Study (CCS-1). Lancet 1995; 345: 686-87.
Califf R M, Woodlief L H. Pragmatic and mechanistic trials. Europ Heart J 1997; 18:367-370.
EuroQol Group. EuroQol a new facility for the measurement of health-related quality of life. Health Policy 1990; 199-208.
Schulman K A, Glick H A, Buxton M, et. al. The economic evaluation of the FIRST study: Design of a prospective analysis alongside a multinational phase III clinical trial. Controlled Clinical Trials 1996; 17:304-315.
Schulman K A, Buxton M, Glick H A, et. al. Results of the economic evaluation of the FIRST study: A multinational prospective economic evaluation. International Journal of Technology Assessment in Health Care 1996; 12(4): 698-713.
Morikawa T, and Yoshida M. A useful testing strategy in Phase III trials: combined test of superiority and test of equivalence. Journal of Biopharmaceutical Statistics 1995; 5; 297-306.
Lan K K G and DeMets D L. Discrete sequential boundaries for clinical trials. Biometrika 1983; 70; 659-663.

The preceding examples illustrate the above-described invention; however, they are not intended to restrict the scope of this invention in any manner. All publications and patents mentioned herein are incorporate by reference in their entirety as if set forth in full herein.

1. A method of treating cardiovascular diseases in a patient following myocardial infarction comprising administering a combination of an angiotensin II receptor blocker (ARB) or a pharmaceutically acceptable salt there and a beta-blocker or a pharmaceutically acceptable salt thereof.

2. A method of reducing the risk of mortality in a patient following myocardial infarction comprising administering a combination of an angiotensin II receptor blocker (ARB) or a pharmaceutically acceptable salt there and a beta-blocker or a pharmaceutically acceptable salt thereof.

3. A method of reducing the risk of stroke in a patient following myocardial infarction comprising administering a combination of an angiotensin II receptor blocker (ARB) or a pharmaceutically acceptable salt there and a beta-blocker or a pharmaceutically acceptable salt thereof.

4. The method of claim 1 which further comprises one or more additional anti-hypertensive agents.

5. The method of claim 4 wherein the additional antihypertensive agent is an angiotensin converting enzyme inhibitor (ACEI) and/or a diuretic.

6. The method of claim 1 wherein the patient is a normotensive patient or a patient whose blood pressure is adequately controlled by administration of an ARB or beta-blocker alone or in combination with an additional anti-hypertensive agent other than an ARB or beta-blocker.

7. The method of claim 2 wherein the myocardial infarction is complicated with left ventricular dysfunction or heart failure.

8. The method of claim 1 wherein the ARB is valsartan or a pharmaceutically acceptable salt thereof.

9. The method of claim 1 wherein the beta-blocker is selected from atenolol, metoprolol and propranolol.

10. The method of claim 1 wherein the ARB is valsartan or a pharmaceutically acceptable salt thereof and the beta-blocker is selected from atenolol, metoprolol and propranolol or a pharmaceutically acceptable salt thereof.

11. (canceled)

12. (canceled)

13. (canceled)

14. (canceled)

15. The method of claim 2 which further comprises one or more additional anti-hypertensive agents.

16. The method of claim 3 which further comprises one or more additional anti-hypertensive agents.

17. The method of claim 5 wherein the additional antihypertensive agent is an angiotensin converting enzyme inhibitor (ACEI) and/or a diuretic.

18. The method of claim 6 wherein the additional antihypertensive agent is an angiotensin converting enzyme inhibitor (ACEI) and/or a diuretic.

19. The method of claim 2 wherein the patient is a normotensive patient or a patient whose blood pressure is adequately controlled by administration of an ARB or beta-blocker alone or in combination with an additional anti-hypertensive agent other than an ARB or beta-blocker.

20. The method of claim 3 wherein the patient is a normotensive patient or a patient whose blood pressure is adequately controlled by administration of an ARB or beta-blocker alone or in combination with an additional anti-hypertensive agent other than an ARB or beta-blocker.

21. The method of claim 3 wherein the myocardial infarction is complicated with left ventricular dysfunction or heart failure.

22. The method of claim 2 wherein the ARB is valsartan or a pharmaceutically acceptable salt thereof.

23. The method of claim 3 wherein the ARB is valsartan or a pharmaceutically acceptable salt thereof.

24. The method of claim 2 wherein the beta-blocker is selected from atenolol, metoprolol and propranolol.

25. The method of claim 3 wherein the beta-blocker is selected from atenolol, metoprolol and propranolol.

26. The method of claim 2 wherein the ARB is valsartan or a pharmaceutically acceptable salt thereof and the beta-blocker is selected from atenolol, metoprolol and propranolol or a pharmaceutically acceptable salt thereof.

27. The method of claim 3 wherein the ARB is valsartan or a pharmaceutically acceptable salt thereof and the beta-blocker is selected from atenolol, metoprolol and propranolol or a pharmaceutically acceptable salt thereof.

28. Use according to claim 3 wherein the ARB is valsartan or a pharmaceutically acceptable salt thereof and the beta-blocker is selected from atenolol, metoprolol and propranolol or a pharmaceutically acceptable salt thereof.
